(19) United States
(12) Patent Application Publication
(10) Pub. No.: US 2025/0340701 A1
(43) Pub. Date: Nov. 6, 2025

(54) DENDRIMER-DELIVERED ALPHA-PARTICLE RADIOTHERAPY FOR TREATMENT OF GLIOBLASTOMA AND OTHER CANCERS IN THE BRAIN

(71) Applicant: The Johns Hopkins University, Baltimore, MD (US)

(72) Inventors: Stavroula SOFOU, Baltimore, MD (US); Kannan RANGARAMANUJAM, Highland, MD (US); Rajiv Ranjit NAIR, Baltimore, MD (US)

(21) Appl. No.: 18/871,895

(22) PCT Filed: Jul. 3, 2023
(86) PCT No.: PCT/US2023/069583

§ 371 Date: Dec. 5, 2024

Related U.S. Application Data
(60) Provisional application No. 63/357,707, filed on Jul. 1, 2022.

(51) Int. Cl. C08G 83/00; A61K 51/06; A61P 35/00

(52) U.S. Cl. CPC C08G 83/003; A61K 51/065; A61P 35/00; A61K 2121/00

(57) ABSTRACT

Dendrimers radiolabeled with an alpha particle emitter, such as actinium-225 (²²⁵Ac), and their use for treating tumors, including glioblastomas, are disclosed.

### BACKGROUND

Glioblastoma is a highly heterogeneous and aggressive brain tumor that arises from abundant non-neuronal glial cells called astrocytes, which provide structural support to tissue and function in maintenance of blood brain barrier, neuron survival, and synapse formation. The malignancy accounts for 15-30% of all adult and pediatric brain tumors. The median survival after diagnosis is from 13 to 73 months, with 5-year survival less than 20%. Survival can be prolonged with surgery, radiotherapy, and chemotherapy. The location of the tumor, however, makes it particularly difficult to treat. Further, adverse consequences to peripheral healthy tissue in the developing brain renders treatment prognosis extremely poor in children. Thus, there is an unmet need for strategies to selectively and effectively kill glioblastoma tumor cells and provide long-lasting remission.

### SUMMARY

In some aspects, the presently disclosed subject matter provides a dendrimer radiolabeled with an alpha particle emitter.

In certain aspects, the alpha particle emitter is selected from actinium-225, astatine-211, lead-212, terbium-149, thorium-227, radium-223, radium-224, bismuth-212, and bismuth-213.

In certain aspects, the dendrimer comprises a G1-G10 generation dendrimer. In particular aspects, the dendrimer comprises a G2-G10 generation dendrimer. In more particular aspects, the dendrimer is selected from a G2 to G6 dendrimer, a G4 to G5 dendrimer, and mixtures thereof.

In certain aspects, the dendrimer comprises one or more surface groups. In particular aspects, the one or more surface groups are selected from a hydroxyl surface group, a glucose surface group, and combinations thereof.

In certain aspects, the dendrimer comprises a polyamidoamine (PAMAM) generation four or generation six particle. In particular aspects, the polyamidoamine generation four or generation six particle comprises a surface group selected from a hydroxyl surface group, a glucose surface group, and combinations thereof.

In certain aspects, the dendrimer further comprises a chelating moiety. In particular aspects, the chelating moiety is dodecane tetraacetic acid (DOTA) or diethylenetriaminepentaacetic acid (DTPA).

In certain aspects, the alpha particle emitter comprises actinium-225 (²²⁵ Ac). In particular aspects, the dendrimer comprises a G1-G10 ²²⁵Ac-DOTA-PAMAM dendrimer. In more particular aspects, the G1-G10 ²²⁵Ac-DOTA-PAMAM dendrimer comprises a G2-G10 ²²⁵Ac-DOTA-PAMAM dendrimer. In more particular aspects, the dendrimer is selected from a G2 to G6 ²²⁵Ac-DOTA-PAMAM dendrimer, a G4 to G5 ²²⁵Ac-DOTA-PAMAM dendrimer, and mixtures thereof.

In certain aspects, the ²²⁵Ac-DOTA-PAMAM dendrimer comprises one or more surface groups. In particular aspects, the one or more surface groups are selected from a hydroxyl surface group, a glucose surface group, and combinations thereof. In more particular aspects, the dendrimer is ²²⁵Ac-DOTA-PAMAM-G4-OH and/or ²²⁵Ac-DOTA-PAMAM-G6-OH

In certain aspects, the radiolabeled dendrimer has a particle size ranging from about 5 nm to about 50 nm. In particular aspects, the particle size has a range from about 5 nm to about 10 nm.

In other aspects, the presently disclosed subject matter provides a method for treating a tumor, the method comprising administering to a subject in need of treatment thereof, a radiolabeled dendrimer disclosed herein.

In certain aspects, the tumor comprises a brain tumor. In particular aspects, the brain tumor comprises a glioblastoma. In certain aspects, the brain tumor comprises a metastasis in the brain. In certain aspects, the subject is an adult. In particular aspects, the subject is a pediatric patient.

In some aspects, the administration of the dendrimer comprises a systemic administration. In particular aspects, the systemic administration comprises an intravenous administration.

In some aspects, the method further comprises administering a therapeutically effective amount of temozolomide (TMZ) in combination with the administration of the dendrimer. In certain aspects, the administration of the therapeutically effective amount of TMZ has a synergistic effect in combination with the administration of the dendrimer for the treating of the tumor. In particular aspects, the method comprises a synergistic effect on suppressing outgrowth or regrowth one or more tumor cells.

In some aspects, an amount of dendrimer taken up by tumor-associated activated macrophages is greater than an amount of dendrimer taken up by resting macrophages.

Certain aspects of the presently disclosed subject matter having been stated hereinabove, which are addressed in whole or in part by the presently disclosed subject matter, other aspects will become evident as the description proceeds when taken in connection with the accompanying Examples and Drawings as best described herein below.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Having thus described the presently disclosed subject matter in general terms, reference will now be made to the accompanying Figures, which are not necessarily drawn to scale, and wherein:

FIG. 1 shows schematics of the presently disclosed approach;

FIG. 2 shows the extent of uptake of dendrimers (small nanoparticles) and of liposomes (large nanoparticles) by activated and by resting (non-activated) macrophages (BV2 microglia). Liposomes were utilized as reference nanoparticles in these studies. Macrophages were activated by addition of Lipopolysaccharide (LPS);

FIG. 3A and FIG. 3B show colony survival of (3A) activated BV2 macrophages and (FIG. 3B) GL261 glioblastoma cancer cells after a 6-hour incubation with free ²²⁵Ac-DOTA (black symbols) and with ²²⁵Ac-radiolabeled dendrimers (red symbols). Error bars correspond to the standard deviation of repeated measurements (n=3-6 samples per condition). The extent of colony survival after exposure to free ²²⁵Ac-DOTA is used as a general indicator of the cells' sensitivity to alpha-particle irradiation independent of the form of the delivery carrier. This information is used to compare radiosensitivities across a wide range of other cells studied in the inventors' lab. Y-axis is logarithmic;

FIG. 4A and FIG. 4B show MRI imaged intracranial GL261 glioblastoma tumors over time in (FIG. 4A) female and (FIG. 4B) male C57BL/6 mice;

FIG. 5 shows survival of Temozolomide-treated mice with intracranial GL261 tumors as compared to non-treated animals. 80 mg/Kg of TMZ was injected intraperitoneally on day 10 post tumor inoculation. *p-value=0.011. Therapy was injected i.p. on day 10 after tumor inoculation;

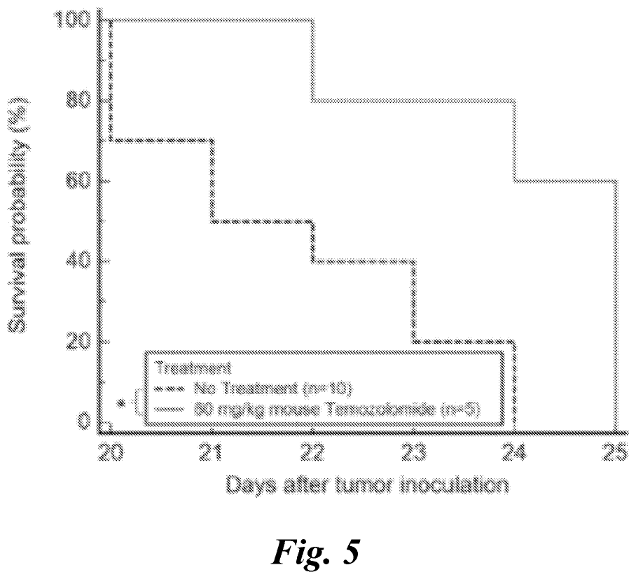

FIG. 6 shows Kaplan-Meier survival plots showing the increased survival of C57BL/6 mice bearing GL-261 glioma tumors when injected intravenously (i.v.) with 600 and/or 700 nCi total radioactivity delivered by the dendrimers. The radiolabeled dendrimers were administered intravenously only once on day 10 post tumor inoculation. *p-value<0.05, **p-value<0.01.

FIG. 7 shows the evaluation of the interstitial pH of GL261 spheroids approximately 400 μm in diameter using the pH indicator SNARF as previously reported (see FIG. S12 in Stras et al., 2016);

FIG. 8 shows (left) Colony Survival fraction of GL261 glioblastoma cells and of resting and/or activated BV2 macrophages exposed to an alpha-particle emitter in the form ²²⁵Ac-DOTA; (right) Colony Survival fraction of GL261 glioblastoma cells and of resting and/or activated BV2 macrophages exposed to an alpha-particle emitter in the form ²²⁵Ac-DOTA-Dendrimer. The mean value of n=3 independent runs are shown. Errors indicate the standard deviations across all independent runs;

FIG. 9 shows uptake of dendrimers by macrophages at 37° C. as a function of incubation time. The incubation conditions are shown in the table embedded in FIG. 9. Cells were incubated with labeled dendrimers and, at different times, a volume sample was removed from the incubating parent suspension, dendrimers not associated with cells were removed by centrifugation, and the fraction of dendrimers associated with cells was quantified and expressed relative to the total concentration of dendrimers in the parent incubation medium. The data show the mean values and standard deviations of n=3 independent measurements;

FIG. 10 shows (left) Colony Survival fraction of GL261 glioblastoma cells exposed to the alpha-particle emitter ²²⁵Ac in the form ²²⁵Ac-DOTA-Dendrimer in the presence and/or absence of Temozolomide (TMZ), at two different concentrations. Surprisingly, colony survival fractions decreased with increasing concentrations of TMZ; (right) killing efficacy (i.e., colony survival fraction) was strongly correlated to the radioactivity associated per cell. Also surprisingly, the extent of radioactivity associated per cell increased with exposure of cells to increasing concentrations of TMZ. The mean value of n=2-3 independent runs are shown. Errors indicate the standard deviations across all independent runs;

FIG. 11 shows the measurement of IC₅₀ of temozolomide (TMZ) on GL261 cells. Cells, plated on monolayers, were incubated with different concentrations of TMZ for 6 hours at 37° C. After completion of incubation, cells were washed, and fresh media were introduced. Following two doubling times of GL261 cells, cells were counted for viability using the MTT assay;

FIG. 12 shows Colony Survival fraction of GL261 glioblastoma cells exposed to an alpha-particle emitter in the form ²²⁵Ac-DOTA in the presence and/or absence of Temozolomide (TMZ) at two different concentrations. Surprisingly, colony survival fractions decreased with increasing concentrations of TMZ. The mean value of n=2-3 independent runs are shown. Errors indicate the standard deviations across all independent runs;

FIG. 13 shows time-integrated radial micro-distributions of fluorescently-labeled dendrimers on GL261 glioblastoma cancer spheroids 400 μm in diameter. The spatial distributions obtained at different timepoints (during carrier uptake by and clearance from spheroids) were integrated using the trapezoid rule along the spheroid radius. Error bars correspond to standard deviations of measurements on n=3 equatorial spheroid sections per time point;

FIG. 14 shows the change of GL261 spheroid volume over time after treatment with radiolabeled dendrimers (²²⁵Ac-DOTA-Dendrimer) and/or TMZ, as described in FIG. 15 (vide infra);

FIG. 15 shows the surprising synergy of ²²⁵Ac-DOTA-Dendrimers when combined with temozolomide (TMZ) on suppressing outgrowth/regrowth of GL261 glioblastoma cells in spheroids, used as surrogates of the avascular regions of solid tumors. Outgrowth/regrowth of GL261 spheroids after incubation with ²²⁵Ac-DOTA-Dendrimers, temozolomide (TMZ), and/or combinations thereof. The concentration of TMZ, when present, was kept constant at 32 μg/mL. Spheroids were incubated with TMZ for 24 hours, to imitate the expected exposure in vivo, where TMZ was administered intraperitoneally. Radiolabeled dendrimers were added in the incubation mixture only for 2 hours (proportional to their blood clearance half-life in vivo). The dendrimer concentration was 10 μg/mL in all conditions, when indicated;

FIG. 16 shows (left): dose fractionation did not effectively alter the survival of treated C57BL/6 mice with intracranial GL261 glioblastoma tumors; (middle): dose response; (right): ²²⁵Ac-DOTA-dendrimers improved survival relative to survival of animals treated with TMZ alone. Surprisingly, combination of ²²⁵Ac-DOTA-dendrimers with TMZ further improved animal survival from all monotherapies (p-value<0.001). TMZ alone, at the injected dose used in these studies, did not result in animal survival different from the non-treated animals with intracranial GL261 tumors; and

FIG. 17 shows H&E stained lung sections from C57BL/6 mice intracranially inoculated with GL261 murine glioblastoma cells. Three levels of magnification of the same lung section are shown for three different treatment types with ²²⁵Ac-DOTA-dendrimer and for one non-treated mouse. Irregular, cancerous masses were observed only in the case of no treatment (bottom right).

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

The presently disclosed subject matter brings together, for the very first time, two unique and independent components: alpha-particle emitters and dendrimer-nanoparticles in an unexpected approach that is appropriate for the treatment of glioblastoma.

The present inventors and others in the field of alpha-particle therapies have been focusing on the engineering of vectors to specifically and directly target certain areas of the tumors and/or the tumors' cancer cells. The presently disclosed approach is fundamentally different because it does not directly aim at the tumors and/or the cancer cells. The presently disclosed approach utilizes another non-tumorigenic cell type, i.e., tumor-associated macrophages, that are in the tumors and have affinity for the dendrimer-nanoparticles, to dynamically infiltrate the tumor. Thus, the presently disclosed approach to alpha-particle therapies departs from the engineering of vectors for direct drug delivery to the engineering of vectors to utilize existing benign cells (cells of the immune system, in this case), which then act as carriers for delivery.

For reasons that are still not entirely clear, dendrimer-nanoparticles have the tendency to accumulate in the brain's tumor-associated macrophages after crossing the Blood Brain-Tumor Barrier. All current research that uses these dendrimer-nanoparticles as carriers of drugs targeted to tumor-associated macrophages aims to either reprogram or kill those cells.

In contrast, the presently disclosed subject matter uses dendrimer-nanoparticles to deliver alpha-particle emitters to the tumor-associated macrophages, but not with the aim to reprogram or kill them. The presently disclosed approach uses those macrophages as the perfect biological tumor infiltrator to selectively and uniformly irradiate and treat glioblastoma, including pediatric glioblastoma.

In some embodiments, the feasibility to stably radiolabel the dendrimer-nanoparticles with alpha-particle emitters at levels high enough to be used as a treatment has been demonstrated and the effective killing of human glioblastoma cells by this approach has been confirmed.

The presently disclosed approach is fundamentally different from clinical and/or preclinical approaches for alpha-particle therapies driven by ‘biochemistry’ (Frontiers in Pharmacology, 2019). Those aim to target certain areas in tumors (vasculature and/or certain receptors on glioblastoma cells) and have failed to elicit long lasting tumor-free responses: this failure is not due to resistance of glioblastoma to alpha-particle therapy, but due to inadequate delivery resulting in non-uniform irradiation of the tumors.

The presently disclosed approach exploits the tumor-associated macrophages in the opposite way of current therapeutic approaches. Unlike current therapeutic approaches with nano drugs that aim to either deplete the tumor associated macrophages or to reprogram them into another form of macrophages that can battle the tumor growth (Frontiers in Immunology, 2019), the presently disclosed approach utilizes the tumor-associated macrophages as intratumoral vehicles to enable tumor infiltration. And, unlike current approaches known in the art, the presently disclosed approach, the greater the population of the tumor-associated macrophages the more extensive an infiltration and more uniform a dispersion will be achieved within the tumor for the alpha-particle emitters.

Macrophages in tumors, also called tumor-associated macrophages, contribute to tumor progression and poor prognosis. In addition, the percentage of tumor-associated macrophages is inversely proportional to the survival period, i.e., higher number of tumor-associated macrophages are correlated with shorter tumor patient survival.

The combination of alpha-particle therapy with dendrimer-nanoparticles delivery can be used to treat glioblastoma, including pediatric glioblastoma, and others cancers of the brain.

The presently disclosed approach opens a new chapter in delivery to brain tumors, including pediatric brain tumors, enabled by identifying the right carrier for the right tumor delivering the right drug trafficked by existing biological processes. If clinically translated, this innovation will provide a viable option to patients, including children, with glioblastoma and the potential for a long lasting, tumor free life.

The presently disclosed approach utilizes both materials (the nanoparticles) and methods (utilizing the tumor-associated macrophages) that are state-of-the-art. This approach is important because of two key characteristics: (1) it can precisely and effectively irradiate and kill tumors within the brain; and (2) it spares the surrounding healthy brain. The irradiation of the surrounding healthy brain is minimal, and this characteristic cannot be achieved by any other type of radiation and/or any other method of delivery. Although the presently disclosed subject matter is directed to tumors in the brain, the approach has the possibility of broader advances: because of its two key characteristics, it may lead to much needed therapeutic interventions against brainstem tumors. Brainstem tumors, for example, being highly innervated, are nearly impossible to overcome effectively. This approach may ultimately address these challenging cases, as well.

More particularly, the presently disclosed subject matter provides an alpha-particle radiotherapeutic specifically tailored for glioblastoma and other cancers in the brain. Alpha-particles are high energy, short-range ionizing particles that kill cells by causing double strand DNA breaks and are impervious to resistance. The short-range of alpha-particles in tissue (only about 5 to 10 cell diameters) assures localized irradiation and killing but, at the same time, it requires a vehicle to distribute the radionuclides uniformly within tumors so as to kill every cell. In alpha-particle radiotherapy, cells not being hit by the alpha-particles will not be killed.

In some embodiments, the presently disclosed subject matter provides a 7-nm diameter dendrimer that is stably radiolabeled with an alpha-particle emitter. In certain embodiments, the alpha particle emitter is selected from actinium-225, astatine-211, lead-212, terbium-149, thorium-227, radium-223, radium-224, bismuth-212, and bismuth-213. In certain embodiments, the alpha-particle emitter comprises Actinium-225 (²²⁵Ac).

In particular embodiments, the radiolabeled dendrimer is injected in the blood of a subject; crosses the Blood Brain Tumor Barrier, but not the Blood Brain Barrier of the healthy brain; and is taken up by the tumor-associated macrophages that reside in the brain tumor. Importantly, the tumor-associated macrophages have the tendency to infiltrate the tumors in the brain. Therefore, while they carry the radioactive dendrimers, they uniformly irradiate the tumors. The radiolabeled dendrimers that are not taken up by the tumor-associated macrophages clear fast from the body.

The presently disclosed subject matter enables selective and uniform irradiation of tumors in the brain, while minimizing the irradiation of the nearby healthy brain. In certain embodiments, the presently disclosed subject matter demonstrates that a single injection of the alpha-particle radiolabeled-dendrimers result in prolonged survival of immune competent mice with intracranial glioblastoma tumors compared to the standard of care.

Accordingly, in some embodiments, the presently disclosed subject matter provides a dendrimer radiolabeled with an alpha particle emitter.

As used herein, the term “dendrimer” refers to repeatedly branched nano-sized macromolecules characterized by a symmetrical (and in some embodiments nonsymmetrical), well-defined three-dimensional shape. Dendrimers grow three-dimensionally by the addition of shells of branched molecules to a central core. The cores are spacious and various chemical units can be attached to points on the exterior of the central core. Dendrimers have been described extensively (Tomalia (1994). Advanced Materials 6:529-539; Donald A. Tomalia, Adel M. Naylor, William A. Goddard III (1990). Angew, Chem. Int. Ed. Engl, 29:138-175; each of which is incorporated herein by reference in its entireties).

Dendrimers can be synthesized as spherical structures typically ranging from about 1 to about 20 nanometers in diameter, including about 1, 2, 3, 4, 5, 6, 7, 8, 9, 10, 11, 12, 13, 14, 15, 16, 17, 18, 19, and 20 nanometers in diameter. In certain embodiments, the dendrimers provided herein have a diameter of from about 1 nm to about 20 nm, such as from about 1 nm to about 8 nm or from about 12 nm to about 20 nm. In certain embodiments, the dendrimer has a diameter of less than or equal to 20 nm, less than or equal to 19 nm, less than or equal to 18 nm, less than or equal to 17 nm, less than or equal to 16 nm, or less than or equal to 15 nm. Diameter may be measured by methods known within the art, such as (but not limited to) dynamic light scattering and electron microscopy.

Dendrimers are identified by a generation number (Gn) and each complete synthesis reaction results in a new dendrimer generation. Molecular weight and the number of terminal (e.g., surface) groups increase exponentially as a function of generation number (e.g., the number of layers) of the dendrimer. Further description of dendrimers can be found in U.S. Pat. No. 9,345,781, WO WO2009/046446, and U.S. Patent Application Publication No. 2017/0043027, all of which are incorporated by reference herein in their entirety.

In some embodiments, the dendrimer comprises a PAMAM dendrimer. As used herein, the term “PAMAM dendrimer” refers to poly(amidoamine) dendrimer, which may contain different cores, with amidoamine building blocks. The method for making them is known to those of skill in the art and generally, involves a two-step iterative reaction sequence that produces concentric shells (generations) of dendritic β-alanine units around a central initiator core. This PAMAM core-shell architecture grows linearly in diameter as a function of added shells (generations). Meanwhile, the surface groups amplify exponentially at each generation according to dendritic-branching mathematics. An exemplary surface group for the disclosed dendrimers is a-OH group. The dendrimers can be generations G1-10 with 5 different core types and 10 functional surface groups. The dendrimer may be of G2 to G10 in range, such as G2 to G6 or G4 to G5, with mixtures of different G levels also possible.

In certain embodiments, the dendrimer comprises one or more surface groups. In particular embodiments, the one or more surface groups are selected from a hydroxyl surface group, a glucose surface group, and combinations thereof.

In certain embodiments, the dendrimer comprises a polyamidoamine (PAMAM) generation four or generation six particle. In particular embodiments, the polyamidoamine generation four or generation six particle comprises a surface group selected from a hydroxyl surface group, a glucose surface group, and combinations thereof.

More particularly, International PCT patent application publication no. WO2016025741 for Selective Dendrimer Delivery to Brain Tumors to Mangraviti et al. (hereinafter Mangraviti et al.), published Feb. 18, 2016, which is incorporated herein by reference in its entirety, describes a composition comprising poly(amidoamine) (PAMAM) hydroxyl-terminated dendrimers covalently linked to or complexed with at least one therapeutic agent for the treatment or alleviation of one or more symptoms of a brain tumor.

In representative embodiments described by Mangraviti et al, the composition contains one or more ethylene diamine-core poly(amidoamine) (PAMAM) hydroxyl-terminated generation-4, 5, 6, 7, 8, 9, or 10 (G4-10-OH) dendrimers. The G6 dendrimers demonstrated unexpectedly high uptake, and uniform distribution in to the entire brain tumor. The dendrimers provided a means for selective delivery through the blood brain barrier (“BBB”) of, for example, chemotherapeutic agents.

The dendrimers may be administered alone by intravenous injection, or as part of a multi-prong therapy with radiation and/or surgery. The dendrimer composition is preferably administered systemically, most preferably via intravenous injection. The composition may be administered prior to or immediately after surgery, radiation, or both. The composition may be designed for treatment of specific types of tumors, such as gliomas, or through targeting tumors associated with microglia/macrophages (TAM).

Mangraviti et al. demonstrated that hydroxyl terminated PAMAM dendrimers demonstrate unique favorable pharmacokinetic characteristics in a glioblastoma tumor model following systemic administration. Dendrimers rapidly accumulate and are selectively retained in the tumor tissue. This is due at least in part to the small size and near neutral surface charge which allow homogeneous distribution of the dendrimer through the entire solid tumor. Dendrimers homogeneously distribute through the extracellular matrix reaching the entire tumor and peritumoral area. Dendrimers intrinsically target neuroinflammation and accumulate in the tumor associated microglia/macrophages (TAMs). Increasing the generation of dendrimers from 4 to 6 can significantly increase dendrimer accumulation in the tumor without affecting their homogeneous distribution and targeting of TAMs. The generation 4 and 6 hydroxyl terminated PAMAM dendrimers can leak through the blood brain tumor barrier and selectively accumulate in glioblastoma, not the peritumoral area, following systemic administration. However, the dendrimers also accumulate in the peritumoral area, thereby having an effect on the migrating front of glioblastoma. These dendrimers intrinsically target tumor associated microglia/macrophages and are retained in these cells over at least 48 hours. There is no significant accumulation of dendrimers in the contralateral hemisphere (‘healthy’) where the dendrimers remain in the blood vessel lumen.

Mangraviti et al. further demonstrated that generation 4 (G4) dendrimers rapidly and selectively accumulate and are retained in the tumor tissue despite their rapid clearance from the circulation. Based on fluorescence quantification and high resolution fluorescence microscopy dendrimers accumulate over the first 8 hours and are still retained in the tumor at 48 hours. Increasing the generation of dendrimers from 4 to 6 can significantly increase dendrimer accumulation, AUC, and retention in the tumor approximately 100-fold without affecting their homogeneous distribution and targeting of TAMs.

In some embodiments, the dendrimer further includes a chelating moiety. Representative chelating moieties include, but are not limited to, the following:

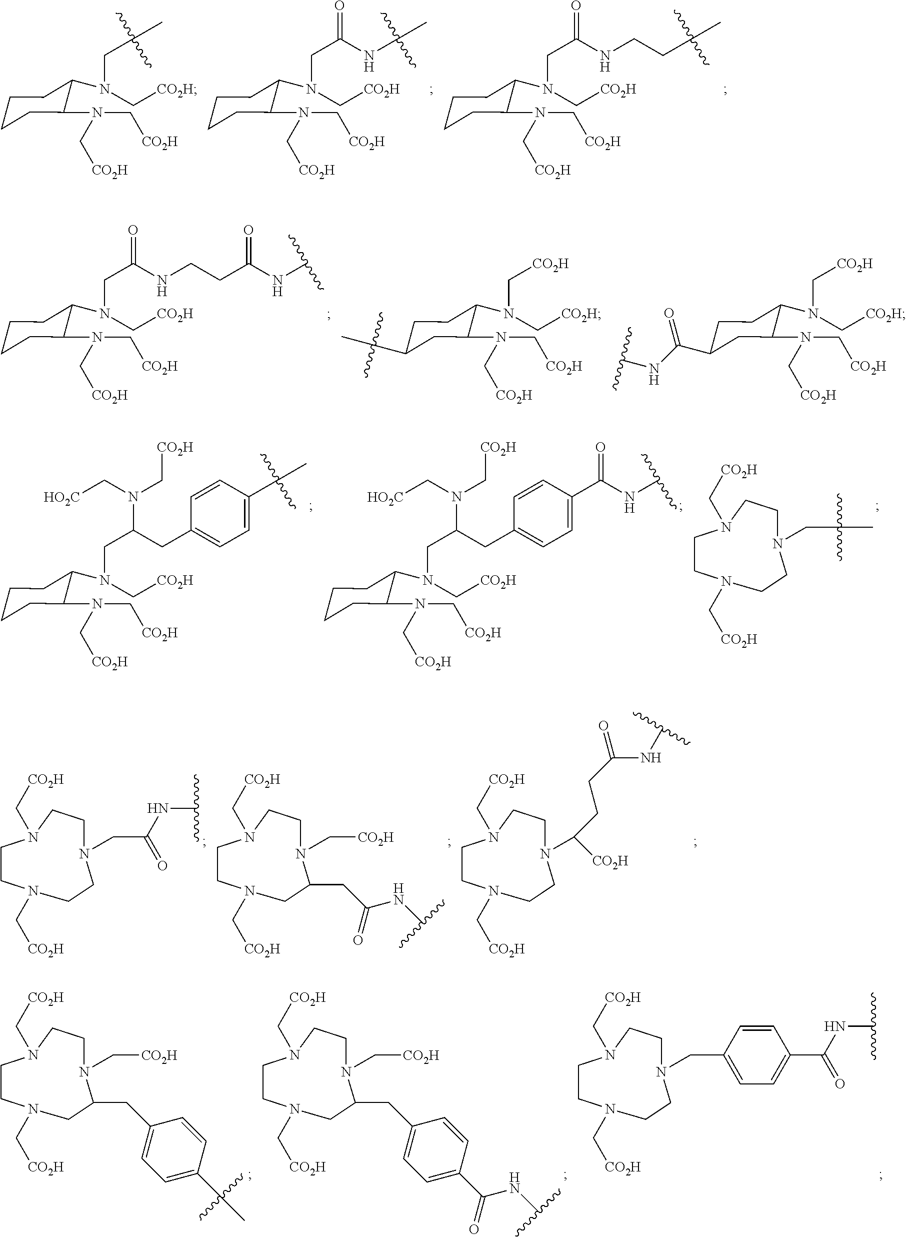

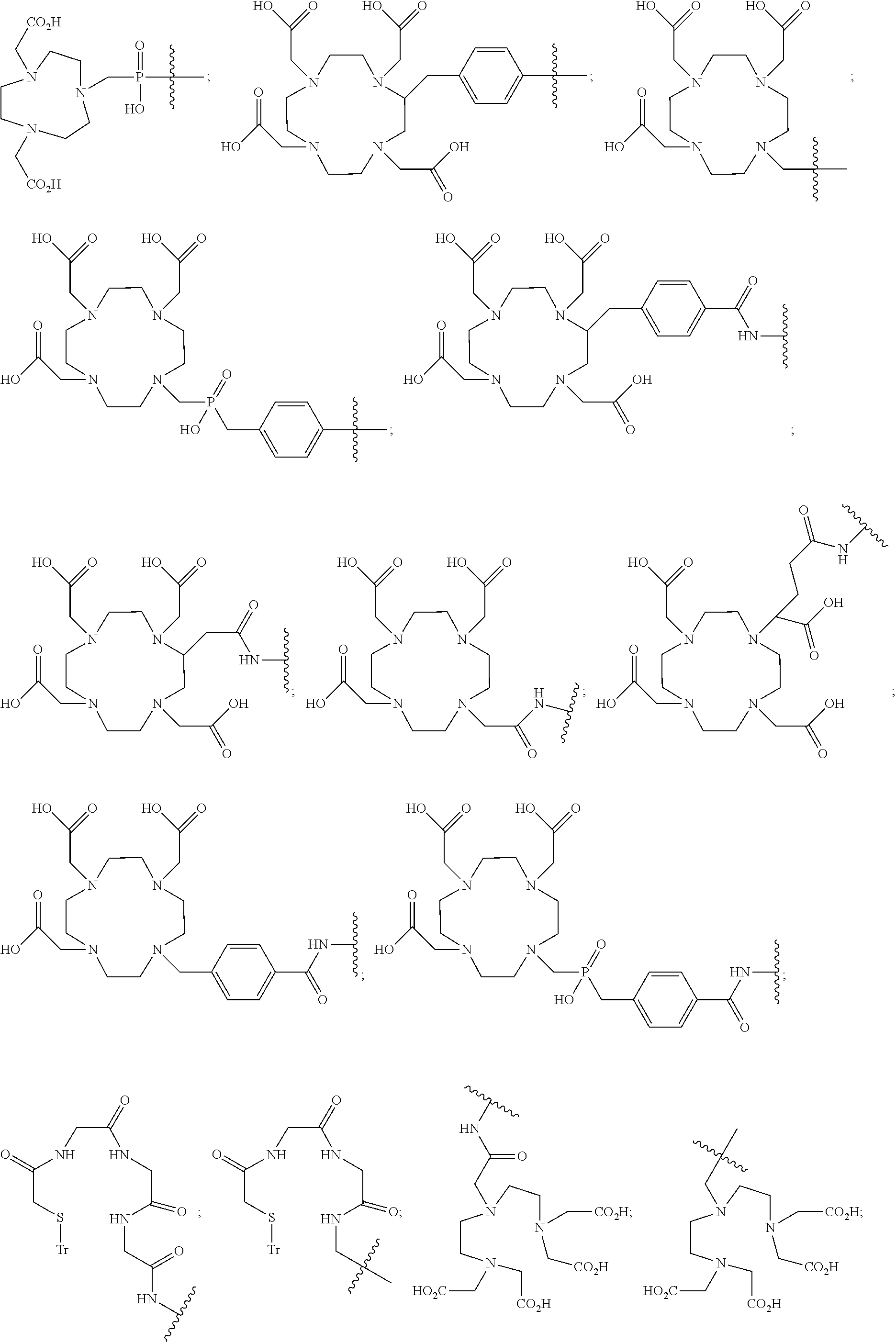

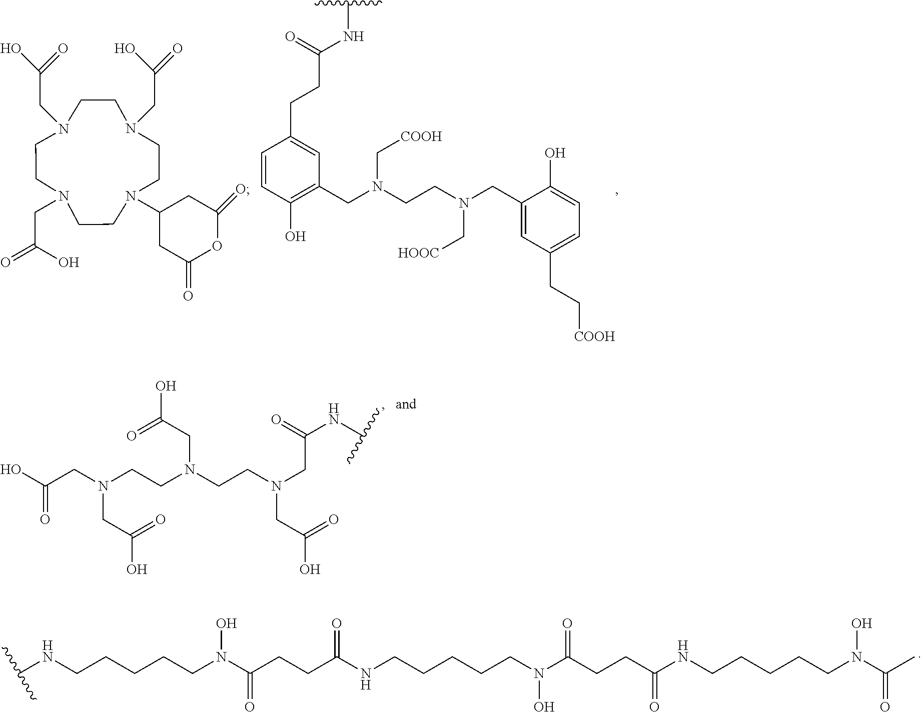

In some embodiments, the chelating moiety is selected from the group consisting of DOTAGA (1,4,7,10-tetraazacyclododececane, 1-(glutaric acid)-4,7,10-triacetic acid), DOTA (1,4,7,10-tetraazacyclododecane-1,4,7,10-tetraacetic acid), DOTASA (1,4,7,10-tetraazacyclododecane-1-(2-succinic acid)-4,7,10-triacetic acid), CB-DO2A (10-bis(carboxymethyl)-1,4,7,10-tetraazabicyclo[5.5.2]tetradecane), DEPA (7-[2-(Bis-carboxymethylamino)-ethyl]-4,10-bis-carboxymethyl-1,4,7,10-tetraaza-cyclododec-1-yl-acetic acid)), 3p-C-DEPA (2-[(carboxymethyl)][5-(4-nitrophenyl-1-[4,7,10-tris(carboxymethyl)-1,4,7,10-tetraazacyclododecan-1-yl]pentan-2-yl)amino]acetic acid)), TCMC (2-(4-isothiocyanotobenzyl)-1,4,7,10-tetraaza-1,4,7,10-tetra-(2-carbamonyl methyl)-cyclododecane), oxo-DO3A (1-oxa-4,7,10-triazacyclododecane-5-S-(4-isothiocyanatobenzyl)-4,7,10-triacetic acid), p-NH2-Bn-Oxo-DO3A (1-Oxa-4,7,10-tetraazacyclododecane-5-S-(4-aminobenzyl)-4,7,10-triacetic acid), TE2A ((1,8-N,N′-bis-(carboxymethyl)-1,4,8,11-tetraazacyclotetradecane), MM-TE2A, DM-TE2A, CB-TE2A (4,11-bis(carboxymethyl)-1,4,8,11-tetraazabicyclo[6.6.2]hexadecane), CB-TE1A1P (4,8,11-tetraazacyclotetradecane-1-(methanephosphonic acid)-8-(methanecarboxylic acid)), CB-TE2P (1,4,8,11-tetraazacyclotetradecane-1,8-bis(methanephosphonic acid), TETA (1,4,8,11-tetraazacyclotetradecane-1,4,8,11-tetraacetic acid), NOTA (1,4,7-triazacyclononane-N,N′,N″-triacetic acid), NODA (1,4,7-triazacyclononane-1,4-diacetate); NODAGA (1,4,7-triazacyclononane, 1-glutaric acid-4,7-acetic acid); NOTAGA (1,4,7-triazonane-1,4-diyl)diacetic acid); DFO (Desferoxamine), NETA ([4-[2-(bis-carboxymethylamino)-ethyl]-7-carboxymethl-[1,4,7]triazonan-1-yl}-acetic acid), TACN-TM (N,N′,N″, tris(2-mercaptoethyl)-1,4,7-triazacyclononane), Diamsar (1,8-Diamino-3,6,10,13,16,19-hexaazabicyclo(6,6,6)eicosane, 3,6,10,13,16,19-Hexaazabicyclo[6.6.6]eicosane-1,8-diamine), Sarar (1-N-(4-aminobenzyl)-3,6,10,13,16,19-hexaazabicyclo[6.6.6]eicosane-1,8-diamine), AmBaSar (4-((8-amino-3,6,10,13,16,19-hexaazabicyclo[6.6.6]icosane-1-ylamino)methyl)benzoic acid), and BaBaSar.

In particular embodiments, the chelating moiety is dodecane tetraacetic acid (DOTA) or diethylenetriaminepentaacetic acid (DTPA).

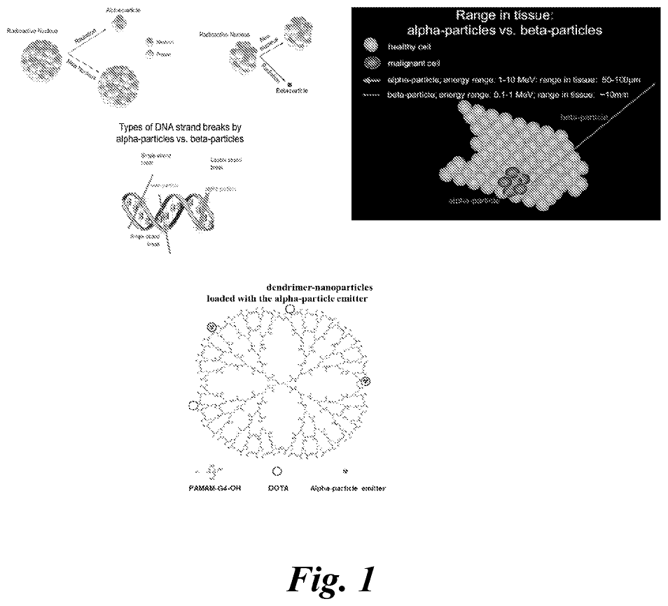

In particular embodiments, the dendrimer nanoparticles comprise PAMAM-G4-OH and/or PAMAM-G6-OH, which are hydroxyl-polyamidoamine, generation-four and/or six (G4 and/or G6) particles. DOTA: 1,4,7,10-tetraazacyclododecane-1,4,7,10-tetraacetic acid is an established chelator for the proposed alpha-particle emitters Actinium-225 and/or Bismuth-213. In such embodiments, the chelated radionuclide is not covalently bound to DOTA, but rather stably retained mostly by attractive electrostatic forces. DOTA-labeled dendrimer-nanoparticles are dendrimer-nanoparticles that are covalently modified with DOTA that is used to chelate the proposed alpha-particle radionuclide. A representative DOTA-labeled generation-four dendrimer is shown in FIG. 1.

In certain embodiments, the alpha particle emitter is selected from actinium-225 (²²⁵Ac), astatine-211 (²¹¹At), lead-212 (²¹²Pb), terbium-149 (¹⁴⁹Tb), thorium-227 (²²⁷Th), radium-223 (²²³Ra), radium (²²⁴Ra), bismuth-212 (²¹²Bi), and bismuth-213 (²¹³Bi). In particular embodiments, the alpha particle emitter comprises actinium-225 (²²⁵Ac).

In particular embodiments, the dendrimer comprises a G1-G10 ²²⁵Ac-DOTA-PAMAM dendrimer. In more particular embodiments, the G1-G10 ²²⁵Ac-DOTA-PAMAM dendrimer comprises a G2-G10 ²²⁵Ac-DOTA-PAMAM dendrimer. In more particular embodiments, the dendrimer is selected from a G2 to G6 ²²⁵Ac-DOTA-PAMAM dendrimer, a G4 to G5 ²²⁵Ac-DOTA-PAMAM dendrimer, and mixtures thereof.

In certain embodiments, the ²²⁵Ac-DOTA-PAMAM dendrimer comprises one or more surface groups. In particular embodiments, the one or more surface groups are selected from a hydroxyl surface group, a glucose surface group, and combinations thereof.

In certain embodiments, the dendrimer is ²²⁵Ac-DOTA-PAMAM-G4-OH and/or ²²⁵Ac-DOTA-PAMAM-G6-OH

In certain embodiments, the radiolabeled dendrimer has a particle size ranging from about 5 nm to about 50 nm, including about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50 nm. In particular embodiments, the particle size has a range from about 5 nm to about 10 nm, including about 5, 6, 7, 8, 9, and 10 nm. In even more particular embodiments, the radiolabeled dendrimer has a particle size of about 7 nm.

In other embodiments, the presently disclosed subject matter provides a method for treating a tumor, the method comprising administering to a subject in need of treatment thereof, a radiolabeled dendrimer disclosed herein.

The term “tumor,” as used herein, refers to an abnormal mass of tissue that results when cells divide more than they should or do not die when they should. In the context of the present disclosure, the term tumor may refer to tumor cells and tumor-associated stromal cells. Tumors may be benign and non-cancerous if they do not invade nearby tissue or spread to other parts of the organism. In contrast, the terms “malignant tumor,” “cancer,” and “cancer cells” may be used interchangeably herein to refer to a tumor comprising cells that divide uncontrollably and can invade nearby tissues. Cancer cells also can spread or “metastasize” to other parts of the body through the blood and lymph systems. The terms “primary tumor” or “primary cancer” refer to an original, or first, tumor in the body. The term “metastasis,” as used herein, refers to the process by which cancer spreads from the location at which it first arose as a primary tumor to distant locations in the body. The terms “metastatic cancer” and “metastatic tumor” refer to the cancer or tumor resulting from the spread of a primary tumor. It will be appreciated that cancer cells of a primary tumor can metastasize through the blood or lymph systems.

In certain embodiments, the tumor comprises a brain tumor. In particular embodiments, the brain tumor comprises a glioblastoma. In certain embodiments, the brain tumor comprises a metastasis in the brain.

In certain embodiments, the subject is an adult. As used herein, an “adult” is a human having an age greater than about 18 years old. In some embodiments, the adult is between 18-50 years old. In other embodiments, the adult is between about 50-100 years old. In other embodiments, the adult is between about 50-75 years old. In other embodiments, the adult is between about 75-85 years old.

In particular embodiments, the subject is a pediatric patient. A pediatric patient can have an age from newborn to about 21 years old. In some embodiments, the pediatric patient is between about 4-9 years old.

Brain tumors generally can be categorized as: primary, starting in the brain; metastatic, starting in other parts of the body and spreading to the brain; benign, slow-growing; non-cancerous. Benign tumors can still be difficult to treat if they are growing in or around certain structures of the brain; and malignant or cancerous. Unlike benign tumors that tend to stay contained, malignant tumors can be very aggressive. They grow rapidly and can spread to areas near the original tumor and to other areas in the brain.

Astrocytomas, including glioblastoma multiforme, are the most common type of glioma, accounting for about half of all childhood brain tumors. They are most common in children between the ages of 5 and 8. The tumors develop from glial cells called astrocytes, most often in the cerebrum (the large upper part of the brain), but also in the cerebellum (the lower back part of the brain).

There are four main types of astrocytomas in children:

Pilocytic astrocytoma (Grade 1): This slow-growing tumor is the most common brain tumor found in children. Pilocytic astrocytoma is often cystic (fluid-filled). When this tumor develops in the cerebellum, surgical removal is often the only treatment necessary. Pilocytic astrocytomas growing in other locations may require other therapies.

Diffuse astrocytoma (Grade 2): This brain tumor infiltrates the surrounding normal brain tissue, making complete surgical removal more difficult. A fibrillary astrocytoma may cause seizures.

Anaplastic astrocytoma (Grade 3): This brain tumor is malignant. Symptoms depend on the location of the tumor. These tumors require a combination of treatments.

Glioblastoma multiforme (Grade 4): This is the most malignant type of astrocytoma. It grows rapidly, and often causes pressure in the brain. These tumors require a combination of treatments.

Other pediatric brain tumors include:
brain stem gliomas: Tumors in this location can be very challenging to treat. Most of these tumors are located in the middle of the brainstem and cannot be surgically removed, particularly the diffuse intrinsic pontine glioma, or DIPG. A few brainstem tumors are more favorably located and can be treated with surgery. These are often treated with non-surgical methods.

Choroid plexus tumors: These tumors are found in the choroid plexus—the part of the brain within the spaces in the brain, called ventricles, that makes cerebrospinal fluid, which surrounds and cushions the brain and the spinal cord. These tumors can cause a buildup of cerebrospinal fluid, resulting in hydrocephalus. They can be benign or malignant, and often require surgery as part of the treatment.

Craniopharyngiomas are benign tumors that occur near the pituitary gland.

Dysembryoplastic neuroepithelial tumors: These rare, benign tumors grow in the tissues covering the brain and spinal cord, and often cause seizures.

Ependymomas are another kind of glioma that forms from the cells that make, support, nourish, and line the ventricles (open areas of the brain's interior that cerebrospinal fluid flows through). They require surgery plus radiation treatment in most cases.

Germ cell tumors: These tumors can be benign or malignant. They grow from germ cells, which form from eggs in women and sperm in men. During normal development of an embryo and fetus, germ cells usually become eggs or sperm.

Medulloblastomas: These malignant brain tumors account for about 15 percent of brain tumors in children. Medulloblastomas form in the cerebellum and occur primarily in children between the ages of 4 and 9, affecting boys more frequently than girls. Medulloblastomas can spread (metastasize) along the spinal cord. They typically require surgery plus other treatments.

Optic nerve gliomas: These tumors are found in or around the optic nerves, i.e., those that send messages from the eyes to the brain. Optic nerve gliomas are frequently seen in children with neurofibromatosis, a type of genetic disorder that affects the skin and nervous system. They can cause vision loss and hormone problems because of their frequent location near the base of the brain. These are typically difficult to treat due to the surrounding sensitive brain structures.

In some embodiments, the administration of the dendrimer comprises a systemic administration. In particular embodiments, the systemic administration comprises an intravenous administration.

In some embodiments, an amount of dendrimer taken up by tumor-associated activated macrophages is greater than an amount of dendrimer taken up by resting macrophages.

As used herein, the term “treating” can include reversing, alleviating, inhibiting the progression of, preventing, or reducing the likelihood of the disease, disorder, or condition to which such term applies, or one or more symptoms or manifestations of such disease, disorder, or condition. Preventing refers to causing a disease, disorder, condition, or symptom or manifestation of such, or worsening of the severity of such, not to occur. Accordingly, the presently disclosed compounds can be administered prophylactically to prevent or reduce the incidence or recurrence of the disease, disorder, or condition.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

In general, the “effective amount” of an active agent or refers to the amount necessary to elicit the desired biological response. As will be appreciated by those of ordinary skill in this art, the effective amount of an agent may vary depending on such factors as the desired biological endpoint, the agent to be delivered, the makeup of the pharmaceutical composition, the drug target, and the like.

Additional therapies that can be used in combination with the presently disclosed ²²⁵Ac-labeled dendrimers include surgery, radiation, including proton therapy, and chemotherapy.

In some embodiments, the method further comprises administering a therapeutically effective amount of temozolomide (TMZ) in combination with the administration of the dendrimer. In certain embodiments, the administration of the therapeutically effective amount of TMZ has a synergistic effect in combination with the administration of the dendrimer for the treating of the tumor. In particular embodiments, the method comprises a synergistic effect on suppressing outgrowth or regrowth one or more tumor cells.

The term “combination” is used in its broadest sense and means that a subject is administered at least two agents, more particularly an ²²⁵Ac-labeled dendrimer nanoparticle in combination with a second therapeutic agent or therapy. More particularly, the term “in combination” refers to the concomitant administration of two (or more) active agents or therapies for the treatment of a single disease state. As used herein, the active agents or therapies may be combined and administered in a single dosage form, may be administered as separate dosage forms at the same time, or may be administered as separate dosage forms that are administered alternately or sequentially on the same or separate days. In one embodiment of the presently disclosed subject matter, the active agents or therapies are combined and administered in a single dosage form. In another embodiment, the active agents or therapies are administered in separate dosage forms (e.g., wherein it is desirable to vary the amount of one but not the other). The single dosage form may include additional active agents therapies for the treatment of the disease state.

Further, the presently disclosed ²²⁵Ac-labeled dendrimer nanoparticle in combination an additional therapeutic agent or therapy can be further administered with adjuvants that enhance stability of the agents, alone or in combination with one or more therapeutic agents, facilitate administration of pharmaceutical compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies.

The timing of administration of the presently disclosed ²²⁵Ac-labeled dendrimer nanoparticle in combination with an additional therapeutic agent or therapy can be varied so long as the beneficial effects of the combination of these agents are achieved. Accordingly, the phrase “in combination with” refers to the administration of an ²²⁵Ac-labeled dendrimer nanoparticle described herein and an additional therapeutic agent or therapy either simultaneously, sequentially, or a combination thereof. Therefore, a subject administered a combination of a presently disclosed ²²⁵Ac-labeled dendrimer nanoparticle and an additional therapeutic agent or therapy can receive an ²²⁵Ac-labeled dendrimer nanoparticle and additional therapeutic agent or therapy at the same time (i.e., simultaneously) or at different times (i.e., sequentially, in either order, on the same day or on different days), so long as the effect of the combination of both agents is achieved in the subject.

When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the ²²⁵Ac-labeled dendrimer nanoparticle and additional agent or therapy are administered simultaneously, they can be administered to the subject as separate pharmaceutical compositions, each comprising either an ²²⁵Ac-labeled dendrimer nanoparticle or at least one additional therapeutic agent, or they can be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

In some embodiments, when administered in combination, the two or more agents can have a synergistic effect. As used herein, the terms “synergy,” “synergistic,” “synergistically” and derivations thereof, such as in a “synergistic effect” or a “synergistic combination” or a “synergistic composition” refer to circumstances under which the biological activity of a combination of a compound described herein and at least one additional therapeutic agent is greater than the sum of the biological activities of the respective agents when administered individually.

Synergy can be expressed in terms of a “Synergy Index (SI),” which generally can be determined by the method described by F. C. Kull et al., Applied Microbiology 9, 538 (1961), from the ratio determined by:

$${Q}_{a} / {Q}_{A} + B / {Q}_{B} = Synergy Index ( SI )$$

wherein:
Q_{A} is the concentration of a component A, acting alone, which produced an end point in relation to component A;
Qₐ is the concentration of component A, in a mixture, which produced an end point;
Q_{B} is the concentration of a component B, acting alone, which produced an end point in relation to component B; and
Q_{b} is the concentration of component B, in a mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount, or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments±50%, in some embodiments±20%, in some embodiments±10%, in some embodiments±5%, in some embodiments±1%, in some embodiments±0.5%, and in some embodiments±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Curing Glioblastoma by Alpha-Particle Radionuclide Therapy

### 1.1 Background

Glioblastoma is a highly heterogeneous and aggressive brain tumor. It arises from abundant non-neuronal glial cells called astrocytes, which provide structural support and play essential functions in maintenance of the blood brain barrier, neuron survival and synapse formation. The malignancy accounts for 15-30% of all adult and pediatric brain tumors. Survival can be prolonged with surgery, radiotherapy, and chemotherapy but the location of the tumor is difficult to treat and adverse consequences to peripheral healthy tissue in the brain can render treatment prognosis extremely poor. With a 5-year survival of less than 20%, patients with glioblastoma have few options. Hence, there is an unmet need for a therapeutic strategy that will selectively and effectively kill glioblastoma cancer cells, an intervention that will deliver long-lasting remission, improve quality of life, and prolong survival.

Radiation therapy uses high-energy particles or waves (x-rays, gamma rays, electron beams, or protons) delivered either by external beam, a source placed inside the body, or systemically by radiopharmaceuticals. One particularly effective form of internal radiation deploys an element with high atomic number that undergoes relatively rapid spontaneous radioactive decay (radionuclide) by emission of alpha particles from its nucleus. A relatively massive amount of energy is deposited by the highly charged alpha particles as they traverse tissue and produce complex double strand breaks in DNA that overwhelm cellular repair in diseased tissue at proximity. The alpha particles have limited penetration in tissue (short travel distance up to 5 to 10 cell diameters), which is an advantage in limiting peripheral damage, but unfortunately, also is related to partial tumor irradiation that is the cause of current glioblastoma treatment failure with alpha-particle therapy.

### 1.2 Scope

To overcome this limitation, the presently disclosed subject matter provides a novel strategy for delivery of alpha particles, based on the systemic administration of a short lifetime alpha emitter attached to 7-nm diameter dendrimer-nanoparticles. The dendrimer-nanoparticles readily cross the blood brain tumor barrier of the tumor brain, but not of healthy brain, and are taken up by tumor-associated macrophages that infiltrate the tumor microenvironment. The nanoparticles localize in activated brain glial cells (macrophages) at a rate proportional to the extent of existing inflammation and that advantageously, clear quickly from the body and brain when not retained inside the tumor-associated macrophages.

The presently disclosed subject matter demonstrates that such dendrimer-nanoparticles loaded with alpha emitters uniformly irradiated the brain tumors at the site of cancer induced inflammation, resulting in significantly better killing efficacy of malignant tumor cells and in longer survival compared to the standard of care and with limited peripheral tissue damage. This strategy, enabled by a nanoparticle platform demonstrated to have an excellent safety profile, represents a new paradigm in radionuclide therapy. Optimization, of dendrimer nanoparticle-based delivery of alpha emitters will effectively kill the cancer cells in glioblastoma and related brain tumors, providing a potential cure, if not just extend remission; a clinical outcome that will improve the quality of life and prolong survival in affected patients.

### 1.3. Preliminary Results

### 1.3.1 Evaluation of Toxicity of the Alpha-Particle Emitter Actinium-225 Delivered by Dendrimer-Nanoparticles

In this embodiment: (i) the efficacy of ²²⁵Ac-dendrimer nanoparticles on tumor cells is quantified; and (ii) the radioactivity levels on dendrimer-nanoparticles tolerated by tumor-associated macrophages is identified.

### 1.3.1.1 Evaluation of the Maximum Tolerated Dose

Tumor-free immunocompetent C57BL/6 mice were intravenously injected with ²²⁵Ac-dendrimers at different radioactivity levels (n=5 animals per radioactivity level). Even at 700 and 800 nCi Actinium-225 labeled-dendrimers per 20 g animal, all animals were alive as of at least 10 months after injection.

### 1.3.1.2 Uptake Extent and Kinetics of Dendrimer-Nanoparticles by Activated and Non-Activated Macrophages

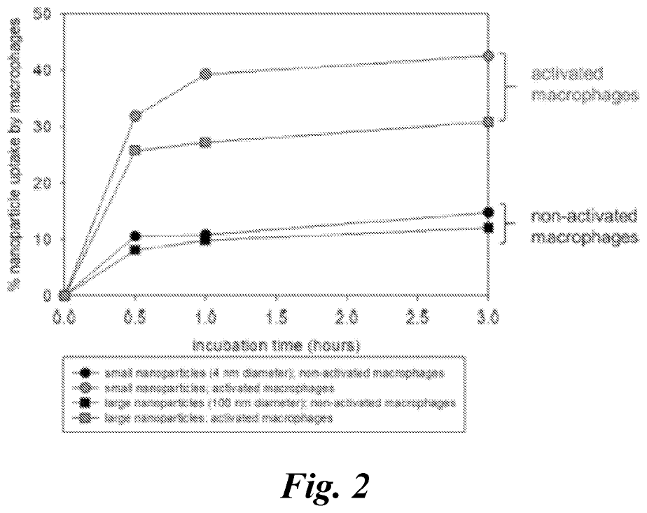

The uptake extent and kinetics of dendrimer-nanoparticles by activated and non-activated macrophages were evaluated (FIG. 2) and were compared to the uptake of larger nanoparticles. Referring now to FIG. 2, tumor-associated macrophages take up dendrimer-nanoparticles significantly more than resting macrophages.

### 1.3.1.3.A Colony Survival Measurement-Murine BV2 Macrophages

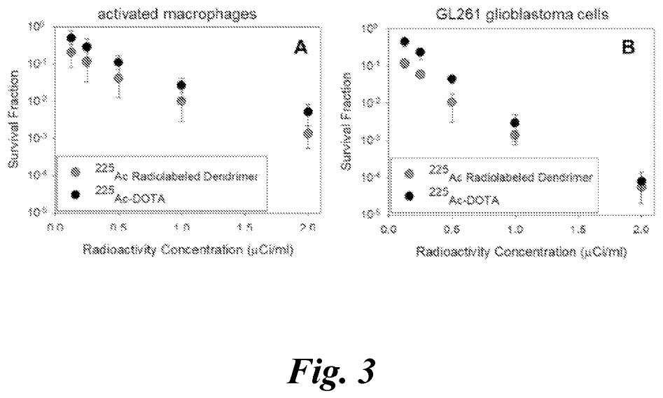

Murine BV2 macrophages were incubated with different radioactivity concentrations of ²²⁵Ac-DOTA dendrimer; cells were plated for colony survival measurement. Results are shown in FIG. 3A.

### 1.3.1.3.B Colony Survival Measurement-GL261 Glioblastoma Cells

GL261 glioblastoma cells were incubated with different radioactivity concentrations of ²²⁵Ac-DOTA dendrimer; cells were plated for colony survival measurement. Results are shown in FIG. 3B. Comparison of the extents of colonies' survival between the macrophages (FIG. 3A) and the glioblastoma cells (FIG. 3B), strongly suggests less sensitivity to alpha-particle radiation of the former compared to the latter type of cells.

Referring once again to FIG. 3A and FIG. 3B, tumor-associated macrophages tolerate higher radioactivity levels on dendrimer-nanoparticles than the GL261 glioblastoma cells in culture (for the same radioactivity concentration, GL261 glioblastoma cells exhibit significantly lower survival than macrophages—note that the y-axis is logarithmic).

Without wishing to be bound to any one particular theory, this result supports the hypothesis that radiation-loaded tumor-associated macrophages may be able to infiltrate the tumors in the brain for, possibly, adequately long periods of time before their own viability is affected by the carried radiation.

### 1.3.2 Assessment of Treatment Efficacy of Actinium-225 Delivered by Dendrimer-Nanoparticles.

### 1.3.2.1 Evaluation of Tumor Growth Kinetics in Different Conditions

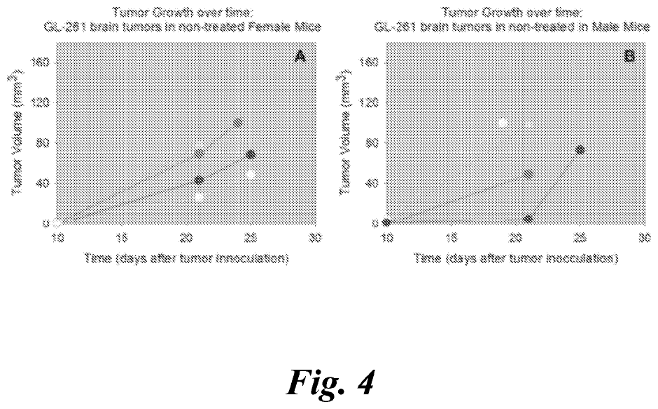

Different numbers of GL261 cells were injected intracranially into the brain of immunocompetent C57BL/6 mice and the growth of tumors was monitored by MRI in the absence of therapy. The aim was to evaluate the tumor growth kinetics in these different conditions, and to understand the limitations of imaging in reporting tumor size. Tumor size was monitored over time on non-treated mice as shown in FIG. 4. This example demonstrates that MRI enables quantitative brain tumor size monitoring in the GL261 brain cancer mouse model.

1.3.3.2 Treatment with Temozolomide

Treatment with the established chemotherapeutic temozolomide (TMZ), the standard of care, was compared to non-treated animals (FIG. 4). Referring now to FIG. 4, temozolomide (TMZ), a standard of care chemotherapeutic, provides survival advantage in the GL261 brain cancer in the immunocompetent C57BL/6 mouse model compared to no treatment.

1.3.3.3 Treatment with Alpha-Particle Dendrimer-Nanoparticles

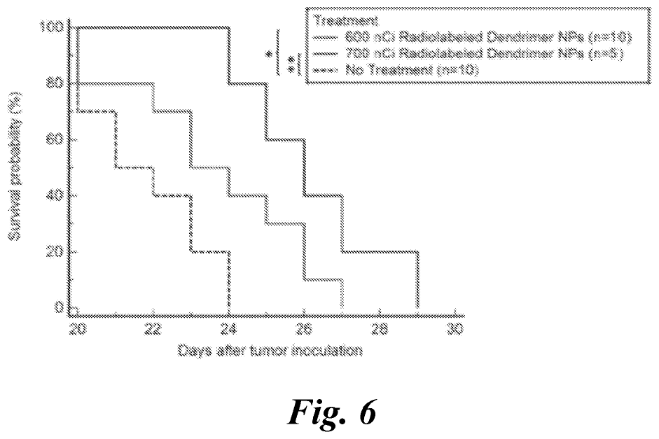

The survival of immune-competent C57BL/6 mice bearing intracranially GL-261 glioma tumors was evaluated upon a single intravenous administration of alpha-particle therapy delivered by the dendrimer carriers for varying total injected radioactivities and was compared to the non-treated condition, as shown in FIG. 6. More particularly, as provided in FIG. 6, a single systemic injection of ²²⁵Ac-labeled dendrimer nanoparticles prolongs survival in the GL261 brain cancer in immunocompetent C57BL/6 mouse model compared to no treatment and to standard of care.

### Example 2

Efficacy of Alpha-Particle Therapy When Delivered by Dendrimers to Prolong Survival of Animals with Intracranial Glioblastoma Tumors

### 2.1 Overview

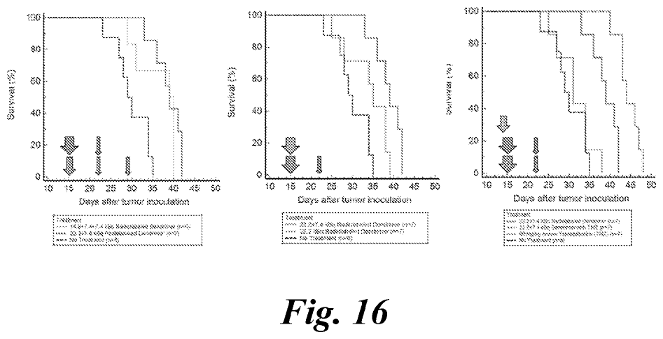

A surprising finding in the results provided in this Example was that when the presently disclosed dendrimer alpha-particle therapy was combined with low doses of the standard of care chemotherapy, the survival was improved even further (see FIG. 16). This improvement was not expected because the killing efficacy of chemotherapy is significantly lower than the efficacy of the presently disclosed dendrimer-radiotherapy. As provided in FIG. 16, chemotherapy alone (gray line on the plot on the right) had no significant effect on survival relative to non-treated animals. Note that in some embodiments, the presently disclosed compositions are injected in the blood (e.g., administered systemically).

### 2.2 Materials and Methods

Ethylenediaminetetraacetic acid (EDTA) was purchased from Fisher Scientific (Pittsburgh, PA, USA), while phosphate buffered saline (PBS) was purchased from Sigma-Aldrich (Atlanta, GA, USA). The 3 kDa MWCO Amicon Ultra-0.5 Centrifugal Filter Unit (Cat. No. UFC5003) was purchased from Milli Pore Sigma (Saint Louis, MO, USA). Matrigel™, Trypsin and the ultra-low adhesion U-shaped 96-well plates (Cat. No.: 7007) were purchased from Corning (Corning, NY, USA), the Dulbecco's Modified Eagle Medium (DMEM) and Roswell Park Memorial Institute (RPMI) medium were from ATCC (Manassas, VA, USA), the Fetal Bovine Serum (FBS) was from Omega Scientific (Tarzana, CA, USA) and penicillin-streptomycin and SNARF-4F were from ThermoFisher Scientific (Waltham, MA, USA). The recombinant murine IL-4 (Cat. No. 214-14) was purchased from Peprotech (Cranbury, NJ, USA). Temozolomide was purchased from Tokyo Chemical Industry (Portland, OR, USA). The 3-(4,5-dimethylthiazol-2-yl)-2,5-diphenyltetrazolium bromide (MTT) assay kit was purchased from Promega (Madison, WI, USA), Chelex® resin from Bio-Rad (Hercules, CA, USA), syringe filters (0.22 μm, Cat No. 76479-024) from VWR (Radnor, PA, USA). Actinium-225 (²²⁵Ac), actinium chloride, was purchased from the U.S. Department of Energy Isotope Program, managed by the Office of Science for Nuclear Physics.

### 2.3 Dendrimer Characterization and Radiolabeling

Hydroxyl terminated generation-6 PAMAM dendrimer nanoparticles (G6-OH) were conjugated with chelators dodecane tetraacetic acid (DOTA or 2,2′,2″,2′″-(1,4,7,10-Tetraazacyclododecane-1,4,7,10-tetrayl)tetraacetic acid) or diethylenetriaminepentaacetic acid (DTPA or (2,2′,2″,2′″-{[(Carboxymethyl)azanediyl]bis(ethane-2,1-diylnitrilo)}tetraacetic acid)). The molecular weight distribution of the conjugated dendrimers was obtained using matrix-assisted laser desorption/ionization technique (MALDI). A Nanoseries Zetasizer (Malvern Instruments Ltd, Worcestershire, UK) was used to evaluate the size distribution and zeta potential of the conjugated dendrimers.

To radiolabel the dendrimer with ²²⁵Ac (or ¹¹¹In), ²²⁵AcCl₃ (or ¹¹¹InCl₃), chelator-conjugated dendrimer was suspended in 500 μL of Tris-HCl buffer, pH 9.0 (or acetate buffer, pH 4.5) that was prepared in water, which was previously passed through Chelex® resin; radioactivity dissolved in 0.2 M HCl was added, and the reaction mixture was incubated at 37° C. for one hour. The dendrimer was then purified via ultra-centrifugation using a 3-kDa molecular weight cut-off (MWCO) ultra-centrifugation filter, washing it 3 times at 12000 rpm for 15 mins each with PBS at 1-mM EDTA, pH 7.4. The radiolabeling efficiency was calculated as the ratio of the measured radioactivity before the first wash and after the final wash. Radiochemical purity was evaluated using iTLC with 10-mM EDTA in water as the mobile phase. McDevitt et al., 2002.

The specific radioactivity of the dendrimer (after reaching secular equilibrium of ²¹³Bismuth≥3.5 hours) was measured by counting the γ-photon emissions of ²¹³Bismuth, 360-480 keV (or ¹¹¹In, 100-400 keV) using a γ-counter (Packard Cobra II Auto-Gamma, Model E5003). The stability of the dendrimer radiolabeling was assessed by adding the radiolabeled dendrimer to media at pH 7.4. Following 24 hours of incubation, the dendrimer was washed using an ultracentrifuge, three times at 12000 rpm for 15 minutes each, and the radioactivity post the final wash was measured. The stability of radiolabeling was then calculated as the ratio of the measured radioactivity before incubation and after the final wash post 24-hour incubation in media.

### 2.4 Cell Lines

The murine glioma cell line, GL261, and the murine BV2 macrophages were cultured using Roswell Park Memorial Institute (RPMI) and Dulbecco's modified Eagle's media (DMEM), respectively, each being supplemented with 10% FBS, 100 units/mL Penicillin and 100-μg/mL Streptomycin in an incubator at 37° C. and 5% CO₂. For the M1 activation of the macrophages, the cells were incubated in half serum media, i.e., DMEM media supplemented with 5% FBS, 100 units/mL Penicillin and 100 μg/mL Streptomycin (PS), containing 100 ng/ml of Lipo-polysaccharide (LPS), in an incubator at 37° C. and 5% CO₂ for 48 hours. For the M2 activation of the macrophages, the cells were incubated in DMEM media supplemented with 10% FBS, 100 units/mL Penicillin and 100 μg/mL Streptomycin, also containing 8 ng/ml of murine IL-4, in an incubator at 37° C. and 5% CO₂ for 48 hours.

### 2.5 IC₅₀ Evaluation

To determine the half-maximal inhibitory concentration (IC₅₀) of temozolomide, a 96-well plate was plated with 20,000 cells per well. A range of concentrations of temozolomide prepared by serial dilutions were added to the wells mixed in media (with 10% FBS and 1% PS) at pH 7.4 and 6.2. Upon the completion of a 6 h exposure, the adherent cells were washed twice with PBS, followed by addition of fresh RPMI 1640 media supplemented with 10% FBS and 1% PS to wells. After two doubling times, a 3-(4,5-dimethylthiazol-2-yl)-2,5-diphenyltetrazolium bromide (MTT) assay was used (following the vendor's instructions) to evaluate percent cell viability. Absorbance was read at 570 nm.

### 2.6 Colony Formation Assay and Radioactivity Per Cell Measurements

Cells, plated at 500,000 cells per well, were plated in 6-well plates and were allowed to adhere overnight before being incubated with varying concentrations of radioactivity (4.63, 9.25, 18.5, 37, 74 kBq/mL) at pH 7.4 for a duration of 6 hours. In case of the combination runs with temozolomide (TMZ), the free drug incubation at respective concentrations, was performed simultaneously with the radioactivity for a duration of 6 hours. Following incubation, the cells in each of the wells were washed with PBS at pH 7.4, gently scraped and resuspended at a concentration of 10,000 cells/mL in media at pH 7.4. Following this step, they were plated into tissue culture dishes at varying cell densities. Once cell colonies were observed (approximately 6 weeks for the cancer cells and approximately 3 weeks for the macrophages), the media was removed, dishes were washed with water, and the colonies were fixed and stained using 6% (w/v) glutaraldehyde and 0.05% (w/v) Crystal violet, respectively, following which they were counted using a colony counter pen. The number of colonies counted for each of the treatment groups was then normalized by the number of colonies from the control group to obtain the survival fraction, while accounting for the plating efficiency. The same procedure was used to obtain the survival fractions for free chemotherapeutic treatment, replacing the radioactivity concentrations with free chemotherapeutic concentration while incubating the cells. Franken et al., 2006.

To measure radioactivity per cell, in each of the colony assay runs, for the highest two radioactivity concentrations, post washing with PBS at pH 7.4, scraping and resuspending, a sample was taken out, left to equilibrate for ≥3.5 hours (to reach secular equilibrium of ²¹³Bismuth), and was then measured by counting the γ-photon emissions of ²¹³Bismuth, 360-480 keV using a γ-counter (Packard Cobra II Auto-Gamma, Model E5003). The sample was then counted for number of cells using Trypan Blue staining method, manually on a hemocytometer. The measured radioactivity from the γ-counter was divided by the cell count to obtain the radioactivity per cell.

### 2.7 Spheroid Formation, Spatiotemporal Distributions, and Treatment

Spheroids were formed by plating 1000 cells/well onto ultra-low adhesion U-shaped 96-well plates (in 1.8% v/v Matrigel™) and centrifuging it at 1023 RCF at 4° C. for 10 minutes, after which they were then left to grow until they reached the desired size of 400 μm in diameter.

The cell density of the spheroids was measured using trypsin when it reached a size of 400 μm in diameter. The spheroids were imaged on Olympus IX80, fished, and incubated in 50 μL of trypsin for 2 hours with repeated mixing every 30 minutes. The number of cells was counted manually on a hemocytometer and the cell density was determined as a ratio of the total number of cells counted to the sum of the spheroid volumes. The experiment was performed in triplicates with 10 spheroids per study.

For the measurement of the interstitial pHe gradient of the spheroids, spheroids at a size of 400±20 μm, were incubated for 12 hours with 200 μM SNARF-4F, a membrane-impermeant pH sensitive indicator (ex: 488 nm, em: 580 nm and 640 nm) whose ratio of fluorescence intensities in the red and the green channels were dependent on the pH. After the incubation, the spheroids were fished and transferred to wells containing fresh media for imaging using a Zeiss LSM 780 Laser Scanning Confocal Microscope. Five z-stacks of 20 μm thickness were obtained for each spheroid to permit identification of the equatorial optical slice on which an in-house erosion algorithm was run to measure the average intensities in both the red and green channel on 3-μm concentric rings from the spheroidal edge to the core. The ring-averaged intensities of spheroid equatorial slices not incubated with cell were subtracted from the fluorescence intensities to remove any background fluorescence contribution. A calibration curve was established by imaging free SNARF-4F in wells containing media (50 μM) of known pH in the range of interest (7.4-6.0) in the red and green channels and was used to determine the corresponding pH for each spheroidal red/green ratio. Stras et al., 2016.

For dendrimer spatiotemporal distributions, spheroids were incubated with PAMAM G6-OH-Cy5 (ex, 651 nm; em, 670 nm) at a final concentration of 10 μg/mL once they reached a size of approximately 400 μm in diameter. Spheroids were incubated for a total of 3 hours to observe the uptake profiles of dendrimers. At the end of the incubation, spheroids were transferred to fresh media to observe clearance profiles. At different time points (0.5 h, 1 h, 2 h and 3 h during uptake and 0.5 h, 1 h and 2 h during clearance), spheroids were fished out in 1-μL media and frozen in Cryochrome™ gel over dry ice. Spheroids that were not incubated with the nanoparticles were used as background. The cryomolds were sliced on the HM550 cryotome at 20-μm thickness and slices close to the center (equatorial slices) were imaged on the Zeiss LSM 780 Laser Scanning Confocal Microscope (Zeiss, White Plains, NJ, USA). The radial distribution of the fluorescence intensities was evaluated by analyzing the images by the erosion code developed in-house by averaging the intensities of concentric rings. A sample of three slices were analyzed for each condition. Fluorescence intensities were converted to concentrations using a calibration curve constructed using serial dilutions of the entities in similar imaging conditions with a quartz 20-μm cuvette.

GL261 spheroids of the approximate size of approximately 400 μm were incubated with different radioactivity concentrations (3, 6, 9 kBq/mL) of dendrimers for 2 hours in a media volume of 150 μL. In case of the treatment group containing temozolomide, the spheroids were incubated with the desired concentration of temozolomide as a standard of care for 24 hours. The incubation times were chosen to agree with the relevant pharmacokinetic clearances in vivo. Post completion of the incubation times, the spheroids were then fished and transferred to fresh media and their growth was monitored by imaging for 10 days. After 10 days of imaging, an outgrowth assay was conducted. Treated and non-treated spheroids were plated on a 96-flat well cell culture treated plates and were allowed to adhere on the flat bottom surface. The spheroid cells were allowed to grow until the spheroidal cells from the non-treatment group reached between 70% to 80% of plate confluency. The cells were then trypsinized, diluted in fresh media and counted manually using a hemocytometer. Results were then normalized to the non-treated measured cell population to obtain a percent spheroid outgrowth value.

### 2.8 Animal Study

Mice were housed using filter top cages with sterile food and water. The animal studies were performed in compliance with Institutional Animal Care and Use Committee protocol (IACUC) guidelines. C57BL/6 mice, 20 g male mice at six weeks of age were purchased from Jackson Laboratory (Bar Harbor, ME, USA). The mice were inoculated with glioblastoma tumors using intracranial injection method following the protocol described in reference. Sharma et al., 2021.

After 9 or 14 days from the day of tumor inoculation, each of the mice was scanned on the Simultaneous 7T Bruker PET-MR Scanner (Brucker, Billerica, MA, USA) and the presence of tumor was confirmed. Following this procedure, the mice with confirmed tumors were randomly assigned to a treatment condition/control group. In the case of the chemo-radioactivity combination treatment groups and the TMZ only group, 80 mg/kg mouse of TMZ was administered intraperitoneally at a volume of 100 μL. On the following day, all the treatment group mice were intravenously administered 100 μL of therapy at a total radioactivity of 22.2 kBq or 14.8 kBq per 20 g animal. For cases with multiple injections, the subsequent injections also were administered at a volume of 100 μL intravenously, at a total radioactivity of 7.4 kBq per 20 g animal each time, 22 and 29 days from the day of tumor inoculation.

Animal weights were monitored daily until the study end point criterion was met. A 25% loss in the weight of the mouse was set as the end point criterion for this study. Upon reaching the end point, the mouse was euthanized, and the tumor as well as other critical organs were harvested and fixed, following which H&E staining was performed. Histological evaluation was then performed on these. The effect of therapy on the prolongation of survival of these tumor bearing mice, compared to the no treatment/control group, was then evaluated by using the Kaplan-Meier plots obtained from the occurrence of the end point events for each of the treatment/control groups.

### 2.9 Statistical Analysis

Results were reported as the arithmetic mean of n independent measurements±the standard deviation. One-way ANOVA and/or post hoc unpaired Student's t test were used to calculate significant differences in efficacy with p-values<0.05 considered to be significant. *indicates 0.01<p-values<0.05; **p-values<0.01.

### 2.10 Results

### 2.10.1 Dendrimer Characterization

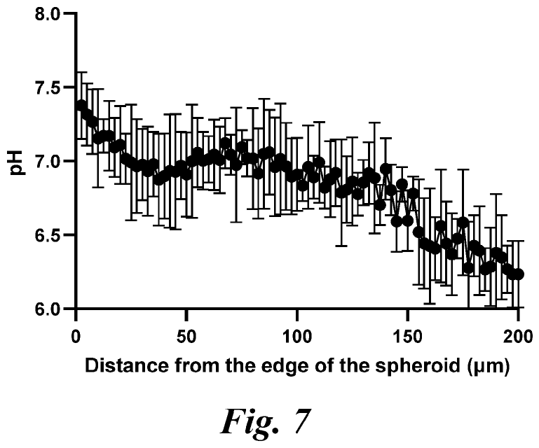

DOTA-functionalized dendrimer nanoparticles (DOTA-dendrimers) were each associated, on average, with four DOTA moieties and were stably radiolabeled with ²²⁵Ac resulting in reasonable levels of specific activities (Table 1). The size of DOTA-dendrimer nanoparticles was approximately 7 nm, and the zeta potential, in PBS, was 0.49±0.03 mV (at pH 7.4) and −0.09±0.12 mV (at pH 6.0, chosen to represent the acidic interstitium in spheroids, FIG. 7).

TABLE 1
Characterization of Dendrimer Radiolabeling.

| | % | Specific | % | |
|---|---|---|---|---|
| | Radio- | Activity | Radio- | |
| ²²⁵Ac-DOTA- | labeling | (kBq/mg | chemical | % |
| D-OH | Efficiency | Dendrimer) | Purity | Stability* |
| n = 14 | 29.1 ± 1.5 | 3885 ± 222 | 94.4 ± 0.6 | 92.6 ± 0.6 |

*Stability assessed in media at pH = 7.4 at t = 24 hrs

### 2.10.2 Characterization of In Vitro Responses on Cell Monolayers

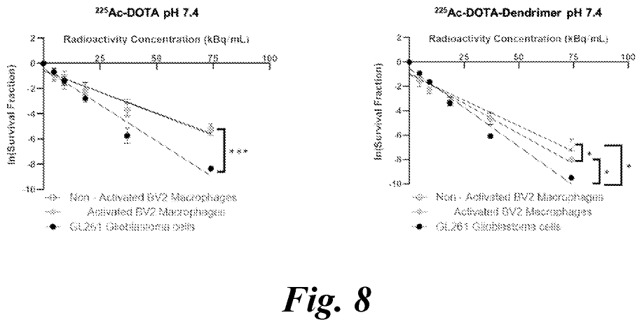

FIG. 8 shows that GL261 murine glioblastoma cells were intrinsically more sensitive to alpha-particle radiation compared to BV2 macrophages. In particular, the colony survival fraction of GL261 cells exposed to ²²⁵Ac-DOTA was significantly lower compared to the survival fraction of colonies formed by resting and activated BV2 macrophages. ²²⁵Ac-DOTA was not expected to associate with any of the cells in a preferential way, Zhu et al., 2017, enabling for comparable irradiation of all cell lines by ²²⁵Ac (i.e., resulting, at each radioactivity concentration, in similar cellular microdoses, whose calculation was beyond the scope of this Example). Without wishing to be bound to any one particular theory, the presently disclosed treatment design was based on the hypothesis that the majority of dendrimer nanoparticles that cross the Blood Brain Tumor Barrier (BBTB) would be taken up by Tumor Associate Macrophages (TAMs).

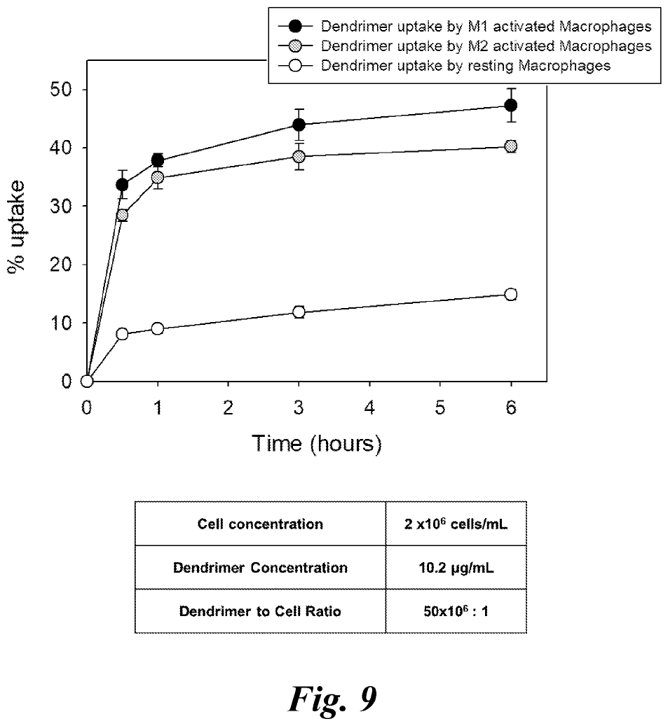

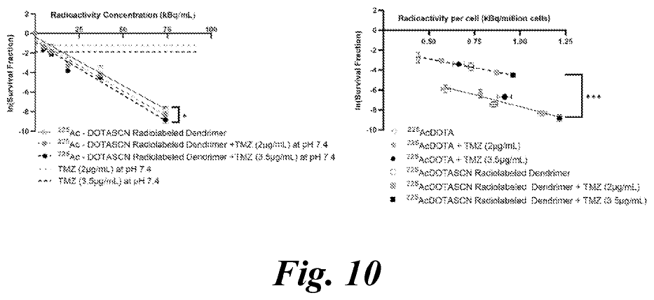

Accordingly, the macrophage sensitivity to ²²⁵Ac-DOTA-dendrimer nanoparticles was evaluated: the difference in the intrinsic response to alpha-particle irradiation, among the activated macrophages and GL261 cells was reduced compared to the response to alpha-particle radiation in the form of ²²⁵Ac-DOTA (shown in FIG. 8, left). This observation could, at least partially, be attributed to the significantly greater uptake of the ²²⁵Ac-DOTA-dendrimer nanoparticles by the activated macrophages compared to their uptake by the GL261 cells (FIG. 9), possibly resulting in higher effective cell “microdoses” to the activated macrophages (vide infra, FIG. 10).

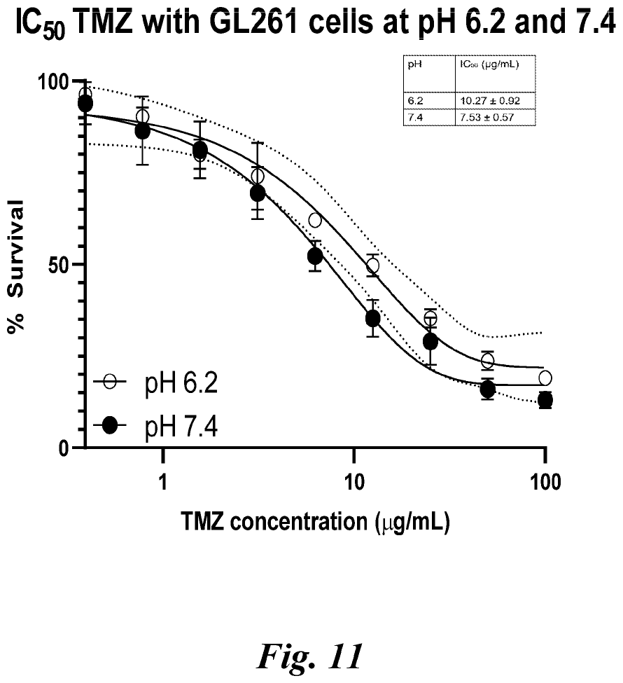

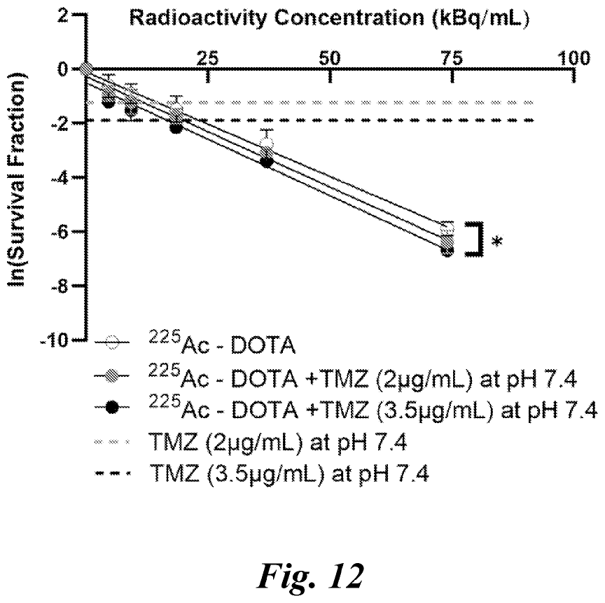

Given that temozolomide (TMZ) is a standard of care for glioblastoma, the effects of ²²⁵Ac on GL261 cells were evaluated in the presence of TMZ. Surprisingly, because the killing effect of chemotherapy is expected to be significantly lower than the effect of alpha-particle therapy, even at relatively low concentrations of TMZ (2.1-3.7 times less than the IC₅₀ at neutral pH, FIG. 11), the GL261 colony survival fractions after irradiation with ²²⁵Ac were lower compared to the survival fractions of cells after irradiation with ²²⁵Ac not exposed to TMZ (FIG. 10, left, for cells exposed to ²²⁵Ac-DOTA-Dendrimer and FIG. 12, for cells exposed to ²²⁵Ac-DOTA). Colony survival fractions were strongly correlated with the levels of radioactivity associated per GL261 cell, independent of the form of ²²⁵Ac (²²⁵Ac-DOTA or ²²⁵Ac-DOTA-dendrimer nanoparticles) and/or the presence of TMZ (FIG. 10, right). Even more surprisingly, when TMZ was present during cell exposure at the same radioactivity concentrations of ²²⁵Ac, greater radioactivity levels per cell were measured (FIG. 10, right), which could be a mechanism partially explaining the enhanced killing of glioblastoma cells in the presence of TMZ, shown in FIG. 10, right.

### 2.10.3 Characterization of In Vitro Responses on 3D Spheroids

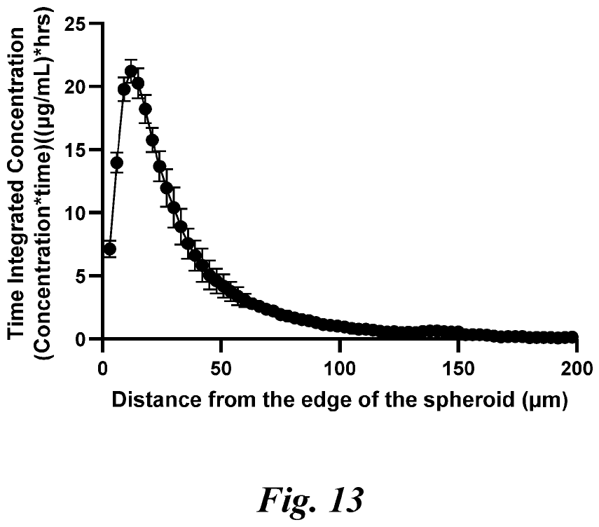

Deep penetration and spreading of dendrimers within glioblastoma tumors, after their systemic administration in vivo, was shown to be due to their uptake by the tumor infiltrating TAMs. Zhang et al., 2015. Correspondingly, in the absence of macrophages in GL261 spheroids, which were utilized as surrogates of the avascular regions of the tumors, the time-integrated radial microdistributions of fluorescently-labeled dendrimers did not demonstrate extensive penetration; instead, significant accumulation was observed within the first 50 μm from the spheroids' edge (FIG. 13).

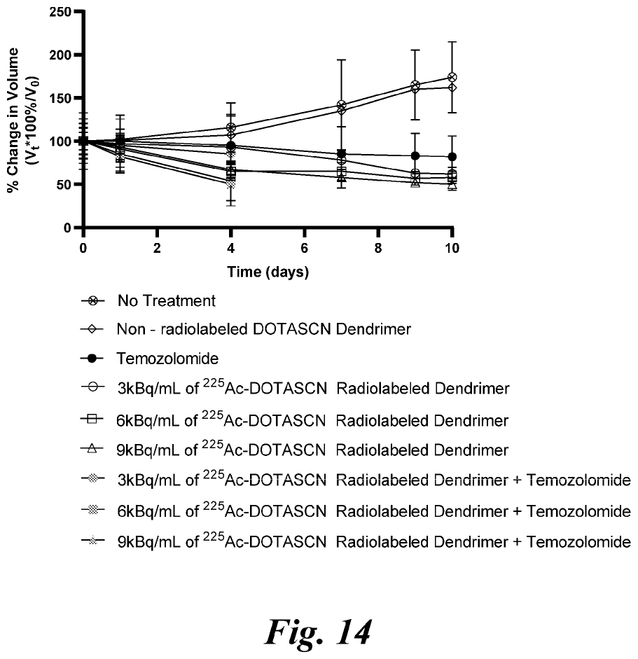

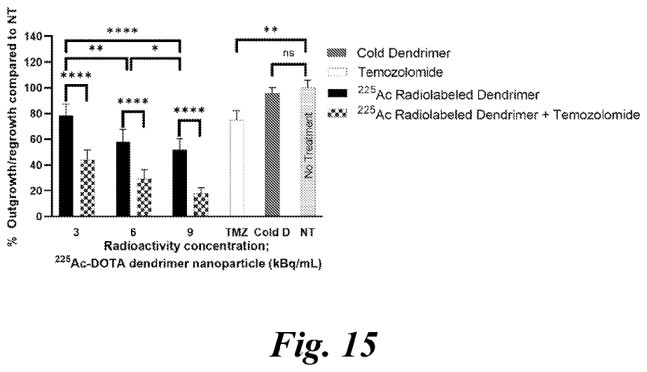

After treatment, the spheroid volume (FIG. 14) and outgrowth/regrowth (used as surrogate of recurrence after treatment) significantly decreased with increasing concentrations of radioactivity in the form of ²²⁵Ac-DOTA-dendrimer nanoparticles (FIG. 15, black bars) relative to no treatment or to spheroids treated with TMZ only). Importantly, the spheroid outgrowth/regrowth was notably further suppressed by addition of TMZ at each level of radioactivity concentration studied (FIG. 15, patterned bar).

### 2.10.4 Response to Treatment

FIG. 16 (left) shows that fractionation of the same total radioactivity of ²²⁵Ac-DOTA-dendrimers, injected intravenously in mice with intracranial GL261 syngeneic tumors, did not significantly affect animal survival, although the time of first death was delayed when a higher fraction of radioactivity was injected initially. Dose response with improved survival relative to no treatment was observed for injected radioactivities equal to and above 22.2 kBq per 20 g mouse (FIG. 16, middle), with the cumulative injected radioactivity of 26.9 kBq per 20 g mouse significantly prolonging survival relative to the lower injected radioactivity (p-value<0.05) and to no treatment (p-value<0.001). The prolonged survival of mice treated with 26.9 kBq of ²²⁵Ac-DOTA-Dendrimer alone was, surprisingly, improved further: although administration of TMZ alone did not have any effect on animal survival relative to no treatment (gray line), co-administration of TMZ and of 26.9 kBq of ²²⁵Ac-DOTA-Dendrimer (dashed red line) resulted in even longer survival compared to the survival of mice that received only radiotherapy (red solid line) (p-value =0.0017) (FIG. 16, right).

### Example 3

### Impact of ²²⁵Ac-DOTA-Dendrimer on Migration and Metastatic Potential of Glioblastoma Cells

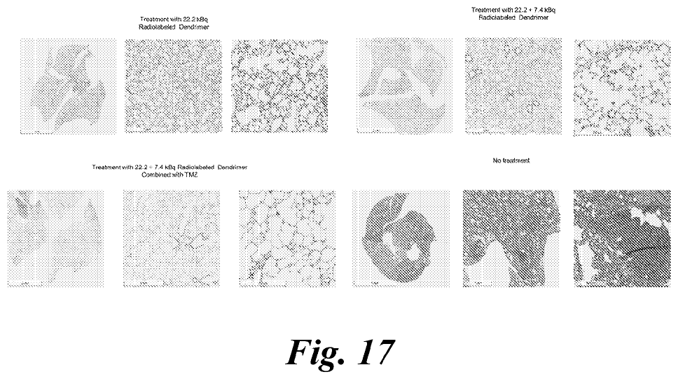

In humans, lung metastasis from glioblastoma is a rare event. But, in the C57BL/6 syngeneic murine model, intracranially inoculated with GL261 murine glioblastoma cells, lung metastases were observed on non-treated tumor-bearing mice (FIG. 17, bottom right). In the same study (for which animal survival is shown on FIG. 16), none of the animals which were injected with alpha-particle therapy, delivered by dendrimers, presented lung metastasis at the time of sacrifice.

The significance of this finding spans beyond the elimination of lung metastases. It should be considered from the perspective of the ability to significantly delay and/or eliminate the migration and/or metastatic potential of glioma cells, in general. In addition, in diffuse gliomas this finding may be of particular significance since the spreading of cancer cells withing the healthy brain, without easily detectable boundaries/low differentiation, is a major factor contributing to the ineffectiveness of current treatment regimens.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents form part of the common general knowledge in the art.
Bailly C, Vidal A, et al. Potential for Nuclear Medicine Therapy for Glioblastoma Treatment. Frontiers in Pharmacology. (10), 2019.
Hu G, Guo M, et al. Nanoparticles Targeting Macrophages as Potential Clinical Therapeutic Agents Against Cancer and Inflammation. Frontiers in Immunology. (10) 1-14, 2019.
McDevitt, M. R., et al., Design and synthesis of 225Ac radioimmunopharmaceuticals. Appl Radiat Isot, 2002. 57 (6): p. 841-7.
Franken, N. A., et al., Clonogenic assay of cells in vitro. Nat Protoc, 2006. 1 (5): p. 2315-9.
Stras, S., et al., Interstitial Release of Cisplatin from Triggerable Liposomes Enhances Efficacy against Triple Negative Breast Cancer Solid Tumor Analogues. Mol Pharm, 2016. 13 (9): p. 3224-33.
Sharma, R., et al., Glycosylation of PAMAM dendrimers significantly improves tumor macrophage targeting and specificity in glioblastoma. J Control Release, 2021. 337: p. 179-192.
Zhu, C., et al., Alpha-particle radiotherapy: For large solid tumors diffusion trumps targeting. Biomaterials, 2017. 130: p. 67-75.
Zhang, F., et al., Uniform brain tumor distribution and tumor associated macrophage targeting of systemically administered dendrimers. Biomaterials, 2015. 52: p. 507-516.

Although the foregoing subject matter has been described in some detail by way of illustration and example for purposes of clarity of understanding, it will be understood by those skilled in the art that certain changes and modifications can be practiced within the scope of the appended claims.

1. A dendrimer radiolabeled with an alpha particle emitter.

2. The dendrimer of claim 1, wherein the alpha particle emitter is selected from actinium-225, astatine-211, lead-212, terbium-149, thorium-227, radium-223, radium-224, bismuth-212, and bismuth-213.

3. The dendrimer of claim 1, wherein the dendrimer is selected from a G1-G10 generation dendrimer, a G2-G10 generation dendrimer, a G2 to G6 dendrimer, a G4 to G5 dendrimer, and mixtures thereof.

4-5. (canceled)

6. The dendrimer of claim 1, wherein the dendrimer comprises one or more surface groups selected from a hydroxyl surface group, a glucose surface group, and combinations thereof.

7-9. (canceled)

10. The dendrimer of claim 1, further comprising a chelating moiety selected from: 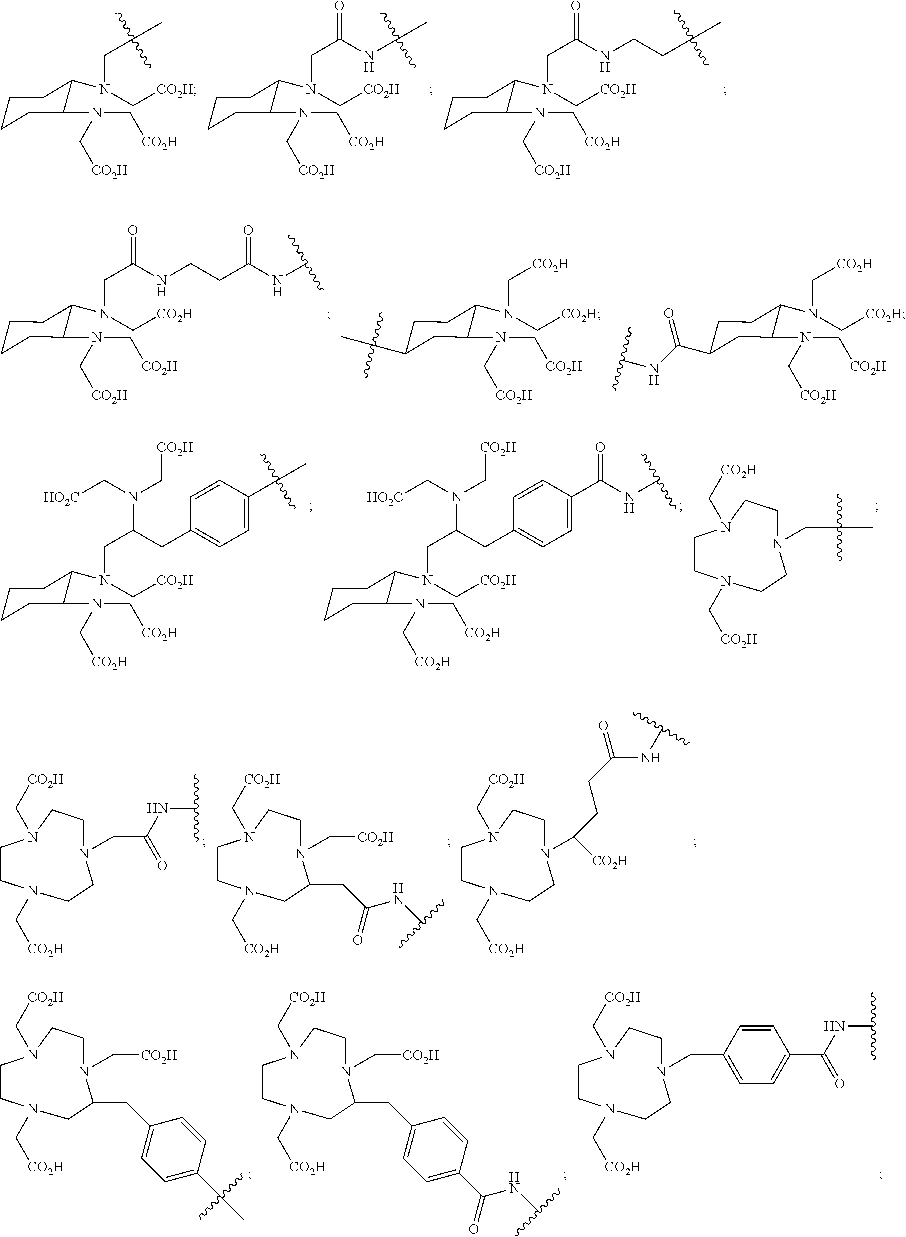 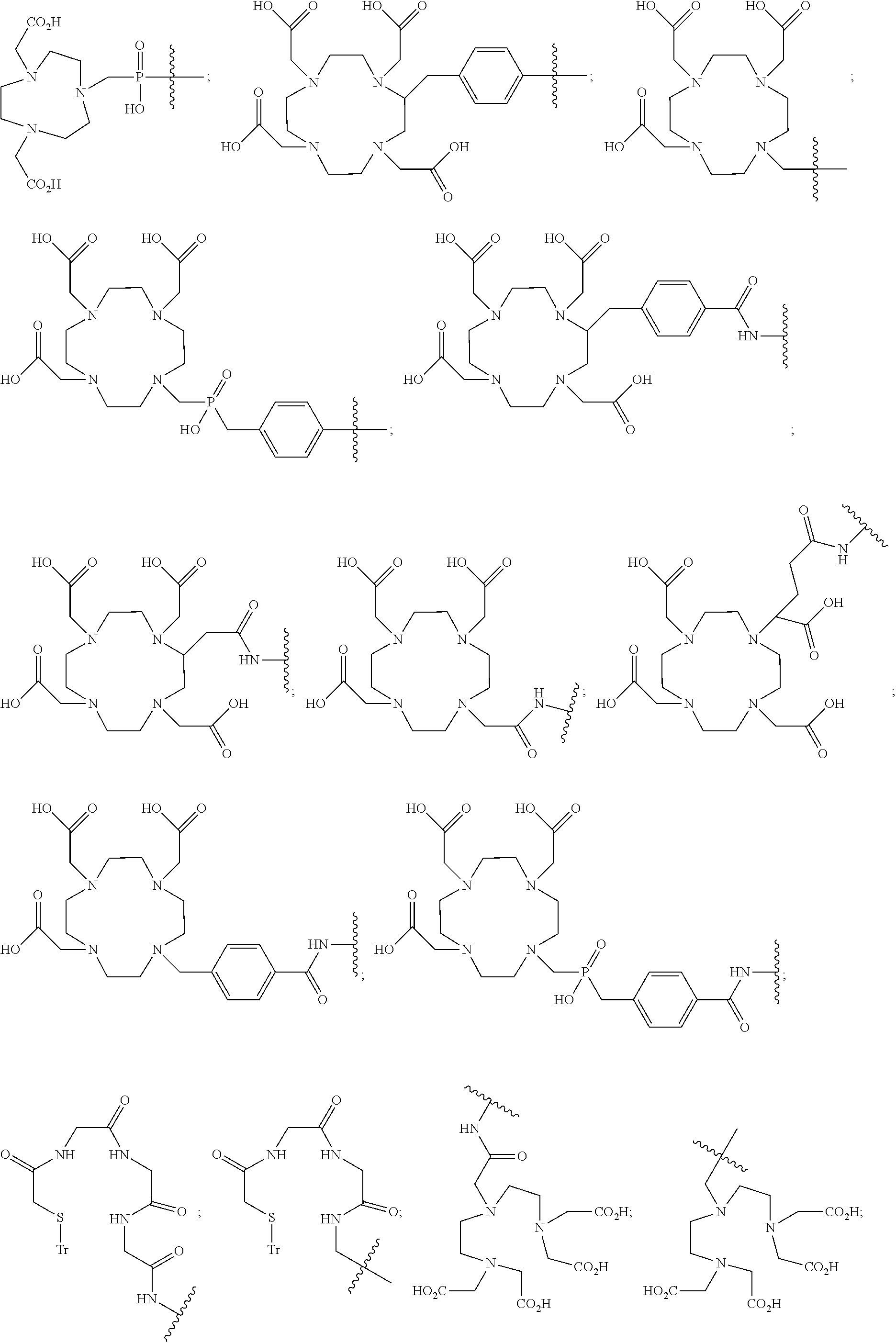 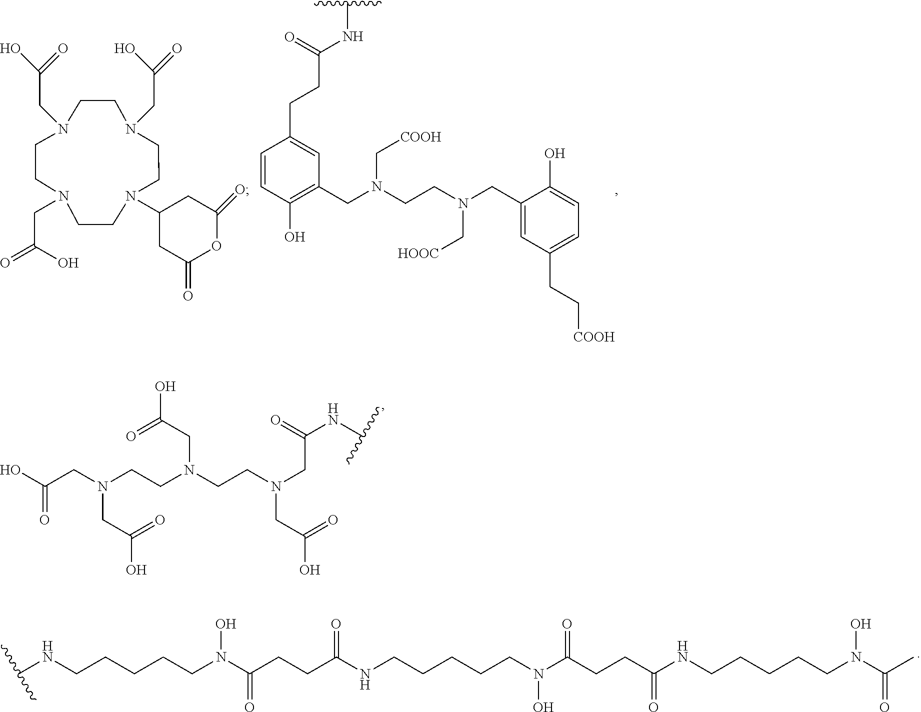
DOTAGA (1,4,7,10-tetraazacyclododececane, 1-(glutaric acid)-4,7,10-triacetic acid), DOTA (1,4,7,10-tetraazacyclododecane-1,4,7,10-tetraacetic acid), DOTASA (1,4,7,10-tetraazacyclododecane-1-(2-succinic acid)-4,7,10-triacetic acid), CB-DO2A (10-bis(carboxymethyl)-1,4,7,10-tetraazabicyclo[5.5.2]tetradecane), DEPA (7-[2-(Bis-carboxymethylamino)-ethyl]-4,10-bis-carboxymethyl-1,4,7,10-tetraaza-cyclododec-1-yl-acetic acid)), 3p-C-DEPA (2-[(carboxymethyl)][5-(4-nitrophenyl-1-[4,7,10-tris(carboxymethyl)-1,4,7,10-tetraazacyclododecan-1-yl]pentan-2-yl)amino]acetic acid)), TCMC (2-(4-isothiocyanotobenzyl)-1,4,7,10-tetraaza-1,4,7,10-tetra-(2-carbamonyl methyl)-cyclododecane), oxo-DO3A (1-oxa-4,7,10-triazacyclododecane-5-S-(4-isothiocyanatobenzyl)-4,7,10-triacetic acid), p-NH2-Bn-Oxo-DO3A (1-Oxa-4,7,10-tetraazacyclododecane-5-S-(4-aminobenzyl)-4,7,10-triacetic acid), TE2A ((1,8-N,N′-bis-(carboxymethyl)-1,4,8,11-tetraazacyclotetradecane), MM-TE2A, DM-TE2A, CB-TE2A (4,11-bis(carboxymethyl)-1,4,8,11-tetraazabicyclo[6.6.2]hexadecane), CB-TE1A1P (4,8,11-tetraazacyclotetradecane-1-(methanephosphonic acid)-8-(methanecarboxylic acid)), CB-TE2P (1,4,8,11-tetraazacyclotetradecane-1,8-bis(methanephosphonic acid), TETA (1,4,8,11-tetraazacyclotetradecane-1,4,8,11-tetraacetic acid), NOTA (1,4,7-triazacyclononane-N,N′,N″-triacetic acid), NODA (1,4,7-triazacyclononane-1,4-diacetate); NODAGA (1,4,7-triazacyclononane,1-glutaric acid-4,7-acetic acid); NOTAGA (1,4,7-triazonane-1,4-diyl)diacetic acid); DFO (Desferoxamine), NETA ([4-[2-(bis-carboxymethylamino)-ethyl]-7-carboxymethl-[1,4,7]triazonan-1-yl}-acetic acid), TACN-TM (N,N′,N″, tris(2-mercaptoethyl)-1,4,7-triazacyclononane), Diamsar (1,8-Diamino-3,6,10,13,16,19-hexaazabicyclo(6,6,6) eicosane, 3,6,10,13,16,19-Hexaazabicyclo[6.6.6]eicosane-1,8-diamine), Sarar (1-N-(4-aminobenzyl)-3, 6,10,13,16,19-hexaazabicyclo[6.6.6]eicosane-1,8-diamine), AmBaSar (4-((8-amino-3,6,10,13,16,19-hexaazabicyclo[6.6.6]icosane-1-ylamino)methyl)benzoic acid), and BaBaSar.

11-12. (canceled)

13. The dendrimer of claim 10, wherein the chelating moiety is dodecane tetraacetic acid (DOTA) or diethylenetriaminepentaacetic acid (DTPA).

14. The dendrimer of claim 1, wherein the alpha particle emitter comprises actinium-225 (²²⁵Ac).

15. The dendrimer of claim 14, wherein the dendrimer is selected from a G1-G10 ²²⁵Ac-DOTA-PAMAM dendrimer, a G2-G10 ²²⁵Ac-DOTA-PAMAM dendrimer, a G2 to G6 ²²⁵Ac-DOTA-PAMAM dendrimer, a G4 to G5 ²²⁵Ac-DOTA-PAMAM dendrimer, and mixtures thereof.

16-19. (canceled)

20. The dendrimer of claim 15, wherein the dendrimer is ²²⁵Ac-DOTA-PAMAM-G4-OH and/or ²²⁵Ac-DOTA-PAMAM-G6-OH.

21. The dendrimer of claim 1 having a particle size ranging from about 5 nm to about 50 nm or about 5 nm to about 10 nm.

22. (canceled)

23. A method for treating a tumor, the method comprising administering to a subject in need of treatment thereof, a dendrimer of claim 1.

24. The method of claim 23, wherein the tumor comprises a brain tumor.

25. The method of claim 24, wherein the brain tumor comprises a glioblastoma.

26. The method of claim 24, wherein the brain tumor comprises a metastasis in the brain.

27. The method of claim 23, wherein the subject is an adult or a pediatric patient.

28. (canceled)

29. The method of claim 23, wherein the administration of the dendrimer comprises a systemic administration.

30. The method of claim 29, wherein the systemic administration comprises an intravenous administration.

31. The method of claim 23, further comprising administering a therapeutically effective amount of temozolomide (TMZ) in combination with the administration of the dendrimer.

32. The method of claim 31, wherein the administration of the therapeutically effective amount of TMZ has a synergistic effect in combination with the administration of the dendrimer for suppressing outgrowth or regrowth of one or more tumor cells.

33. (canceled)

34. The method of claim 23, wherein an amount of dendrimer taken up by tumor-associated activated macrophages is greater than an amount of dendrimer taken up by resting macrophages.
